SEQUENCE LISTING (57 sequences)

SEQ ID NO: 1 - protein, 887 aa, Escherichia coli
MSERFPNDVDPIETRDWLQAIESVIREEGVERAQYLIDQLLAEARKGGVNVAAGTGISNYINTIPVEEQPEYPGNLELERRIRSAIRWNAIMTVLRASKKDLELGGHMASFQSSATIYDVCFNHFFRARNEQDGGDLVYFQGHISPGVYARAFLEGRLTQEQLDNFRQEVHGNGLSSYPHPKLMPEFWQFPTVSMGLGPIGAIYQAKFLKYLEHRGLKDTSKQTVYAFLGDGEMDEPESKGAITIATREKLDNLVFVINCNLQRLDGPVTGNGKIINELEGIFEGAGWNVIKVMWGSRWDELLRKDTSGKLIQLMNETVDGDYQTFKSKDGAYVREHFFGKYPETAALVADWTDEQIWALNRGGHDPKKIYAAFKKAQETKGKATVILAHTIKGYGMGDAAEGKNIAHQVKKMNMDGVRHIRDRFNVPVSDADIEKLPYITFPEGSEEHTYLHAQRQKLHGYLPSRQPNFTEKLELPSLQDFGALLEEQSKEISTTIAFVRALNVMLKNKSIKDRLVPIIADEARTFGMEGLFRQIGIYSPNGQQYTPQDREQVAYYKEDEKGQILQEGINELGAGCSWLAAATSYSTNNLPMIPFYIYYSMFGFQRIGDLCWAAGDQQARGFLIGGTSGRTTLNGEGLQHEDGHSHIQSLTIPNCISYDPAYAYEVAVIMHDGLERMYGEKQENVYYYITTLNENYHMPAMPEGAEEGIRKGIYKLETIEGSKGKVQLLGSGSILRHVREAAEILAKDYGVGSDVYSVTSFTELARDGQDCERWNMLHPLETPRVPYIAQVMNDAPAVASTDYMKLFAEQVRTYVPADDYRVLGTDGFGRSDSRENLRHHFEVDASYVVVAALGELAKRGEIDKKVVADAIAKFNIDADKVNPRLA

SEQ ID NO: 2 - DNA, 2664 bp, Escherichia coli
atgtcagaacgtttcccaaatgacgtggatccgatcgaaactcgcgactggctccaggcgatcgaatcggtcatccgtgaagaaggtgttgagcgtgctcagtatctgatcgaccaactgcttgctgaagcccgcaaaggcggtgtaaacgtagccgcaggcacaggtatcagcaactacatcaacaccatccccgttgaagaacaaccggagtatccgggtaatctggaactggaacgccgtattcgttcagctatccgctggaacgccatcatgacggtgctgcgtgcgtcgaaaaaagacctcgaactgggcggccatatggcgtccttccagtcttccgcaaccatttatgatgtgtgctttaaccacttcttccgtgcacgcaacgagcaggatggcggcgacctggtttacttccagggccacatctccccgggcgtgtacgctcgtgctttcctggaaggtcgtctgactcaggagcagctggataacttccgtcaggaagttcacggcaatggcctctcttcctatccgcacccgaaactgatgccggaattctggcagttcccgaccgtatctatgggtctgggtccgattggtgctatttaccaggctaaattcctgaaatatctggaacaccgtggcctgaaagatacctctaaacaaaccgtttacgcgttcctcggtgacggtgaaatggacgaaccggaatccaaaggtgcgatcaccatcgctacccgtgaaaaactggataacctggtcttcgttatcaactgtaacctgcagcgtcttgacggcccggtcaccggtaacggcaagatcatcaacgaactggaaggcatcttcgaaggtgctggctggaacgtgatcaaagtgatgtggggtagccgttgggatgaactgctgcgtaaggataccagcggtaaactgatccagctgatgaacgaaaccgttgacggcgactaccagaccttcaaatcgaaagatggtgcgtacgttcgtgaacacttcttcggtaaatatcctgaaaccgcagcactggttgcagactggactgacgagcagatctgggcactgaaccgtggtggtcacgatccgaagaaaatctacgctgcattcaagaaagcgcaggaaaccaaaggcaaagcgacagtaatccttgctcataccattaaaggttacggcatgggcgacgcggctgaaggtaaaaacatcgcgcaccaggttaagaaaatgaacatggacggtgtgcgtcatatccgcgaccgtttcaatgtgccggtgtctgatgcagatatcgaaaaactgccgtacatcaccttcccggaaggttctgaagagcatacctatctgcacgctcagcgtcagaaactgcacggttatctgccaagccgtcagccgaacttcaccgagaagcttgagctgccgagcctgcaagacttcggcgcgctgttggaagagcagagcaaagagatctctaccactatcgctttcgttcgtgctctgaacgtgatgctgaagaacaagtcgatcaaagatcgtctggtaccgatcatcgccgacgaagcgcgtactttcggtatggaaggtctgttccgtcagattggtatttacagcccgaacggtcagcagtacaccccgcaggaccgcgagcaggttgcttactataaagaagacgagaaaggtcagattctgcaggaagggatcaacgagctgggcgcaggttgttcctggctggcagcggcgacctcttacagcaccaacaatctgccgatgatcccgttctacatctattactcgatgttcggcttccagcgtattggcgatctgtgctgggcggctggcgaccagcaagcgcgtggcttcctgatcggcggtacttccggtcgtaccaccctgaacggcgaaggtctgcagcacgaagatggtcacagccacattcagtcgctgactatcccgaactgtatctcttacgacccggcttacgcttacgaagttgctgtcatcatgcatgacggtctggagcgtatgtacggtgaaaaacaagagaacgtttactactacatcactacgctgaacgaaaactaccacatgccggcaatgccggaaggtgctgaggaaggtatccgtaaaggtatctacaaactcgaaactattgaaggtagcaaaggtaaagttcagctgctcggctccggttctatcctgcgtcacgtccgtgaagcagctgagatcctggcgaaagattacggcgtaggttctgacgtttatagcgtgacctccttcaccgagctggcgcgtgatggtcaggattgtgaacgctggaacatgctgcacccgctggaaactccgcgcgttccgtatatcgctcaggtgatgaacgacgctccggcagtggcatctaccgactatatgaaactgttcgctgagcaggtccgtacttacgtaccggctgacgactaccgcgtactgggtactgatggcttcggtcgttccgacagccgtgagaacctgcgtcaccacttcgaagttgatgcttcttatgtcgtggttgcggcgctgggcgaactggctaaacgtggcgaaatcgataagaaagtggttgctgacgcaatcgccaaattcaacatcgatgcagataaagttaacccgcgtctggcgtaa

SEQ ID NO: 3 - protein, 630 aa, Escherichia coli
MAIEIKVPDIGADEVEITEILVKVGDKVEAEQSLITVEGDKASMEVPSPQAGIVKEIKVSVGDKTQTGALIMIFDSADGAADAAPAQAEEKKEAAPAAAPAAAAAKDVNVPDIGSDEVEVTEILVKVGDKVEAEQSLITVEGDKASMEVPAPFAGTVKEIKVNVGDKVSTGSLIMVFEVAGEAGAAAPAAKQEAAPAAAPAPAAGVKEVNVPDIGGDEVEVTEVMVKVGDKVAAEQSLITVEGDKASMEVPAPFAGVVKELKVNVGDKVKTGSLIMIFEVEGAAPAAAPAKQEAAAPAPAAKAEAPAAAPAAKAEGKSEFAENDAYVHATPLIRRLAREFGVNLAKVKGTGRKGRILREDVQAYVKEAIKRAEAAPAATGGGIPGMLPWPKVDFSKFGEIEEVELGRIQKISGANLSRNWVMIPHVTHFDKTDITELEAFRKQQNEEAAKRKLDVKITPVVFIMKAVAAALEQMPRFNSSLSEDGQRLTLKKYINIGVAVDTPNGLVVPVFKDVNKKGIIELSRELMTISKKARDGKLTAGEMQGGCFTISSIGGLGTTHFAPIVNAPEVAILGVSKSAMEPVWNGKEFVPRLMLPISLSFDHRVIDGADGARFITIINNTLSDIRRLVM

SEQ ID NO: 4 - DNA, 1893 bp, Escherichia coli
atggctatcgaaatcaaagtaccggacatcggggctgatgaagttgaaatcaccgagatcctggtcaaagtgggcgacaaagttgaagccgaacagtcgctgatcaccgtagaaggcgacaaagcctctatggaagttccgtctccgcaggcgggtatcgttaaagagatcaaagtctctgttggcgataaaacccagaccggcgcactgattatgattttcgattccgccgacggtgcagcagacgctgcacctgctcaggcagaagagaagaaagaagcagctccggcagcagcaccagcggctgcggcggcaaaagacgttaacgttccggatatcggcagcgacgaagttgaagtgaccgaaatcctggtgaaagttggcgataaagttgaagctgaacagtcgctgatcaccgtagaaggcgacaaggcttctatggaagttccggctccgtttgctggcaccgtgaaagagatcaaagtgaacgtgggtgacaaagtgtctaccggctcgctgattatggtcttcgaagtcgcgggtgaagcaggcgcggcagctccggccgctaaacaggaagcagctccggcagcggcccctgcaccagcggctggcgtgaaagaagttaacgttccggatatcggcggtgacgaagttgaagtgactgaagtgatggtgaaagtgggcgacaaagttgccgctgaacagtcactgatcaccgtagaaggcgacaaagcttctatggaagttccggcgccgtttgcaggcgtcgtgaaggaactgaaagtcaacgttggcgataaagtgaaaactggctcgctgattatgatcttcgaagttgaaggcgcagcgcctgcggcagctcctgcgaaacaggaagcggcagcgccggcaccggcagcaaaagctgaagccccggcagcagcaccagctgcgaaagcggaaggcaaatctgaatttgctgaaaacgacgcttatgttcacgcgactccgctgatccgccgtctggcacgcgagtttggtgttaaccttgcgaaagtgaagggcactggccgtaaaggtcgtatcctgcgcgaagacgttcaggcttacgtgaaagaagctatcaaacgtgcagaagcagctccggcagcgactggcggtggtatccctggcatgctgccgtggccgaaggtggacttcagcaagtttggtgaaatcgaagaagtggaactgggccgcatccagaaaatctctggtgcgaacctgagccgtaactgggtaatgatcccgcatgttactcacttcgacaaaaccgatatcaccgagttggaagcgttccgtaaacagcagaacgaagaagcggcgaaacgtaagctggatgtgaagatcaccccggttgtcttcatcatgaaagccgttgctgcagctcttgagcagatgcctcgcttcaatagttcgctgtcggaagacggtcagcgtctgaccctgaagaaatacatcaacatcggtgtggcggtggataccccgaacggtctggttgttccggtattcaaagacgtcaacaagaaaggcatcatcgagctgtctcgcgagctgatgactatttctaagaaagcgcgtgacggtaagctgactgcgggcgaaatgcagggcggttgcttcaccatctccagcatcggcggcctgggtactacccacttcgcgccgattgtgaacgcgccggaagtggctatcctcggcgtttccaagtccgcgatggagccggtgtggaatggtaaagagttcgtgccgcgtctgatgctgccgatttctctctccttcgaccaccgcgtgatcgacggtgctgatggtgcccgtttcattaccatcattaacaacacgctgtctgacattcgccgtctggtgatgtaa

SEQ ID NO: 5 - protein, 474 aa, Escherichia coli [sequence omitted]

SEQ ID NO: 6 - DNA, 1425 bp, Escherichia coli
atgagtactgaaatcaaaactcaggtcgtggtacttggggcaggccccgcaggttactccgctgccttccgttgcgctgatttaggtctggaaaccgtaatcgtagaacgttacaacacccttggcggtgtttgcctgaacgtcggctgtatcccttctaaagcactgctgcacgtagcaaaagttatcgaagaagccaaagcgctggctgaacacggtatcgtcttcggcgaaccgaaaaccgatatcgacaagattcgtacctggaaagagaaagtgatcaatcagctgaccggtggtctggctggtatggcgaaaggccgcaaagtcaaagtggtcaacggtctgggtaaattcaccggggctaacaccctggaagttgaaggtgagaacggcaaaaccgtgatcaacttcgacaacgcgatcattgcagcgggttctcgcccgatccaactgccgtttattccgcatgaagatccgcgtatctgggactccactgacgcgctggaactgaaagaagtaccagaacgcctgctggtaatgggtggcggtatcatcggtctggaaatgggcaccgtttaccacgcgctgggttcacagattgacgtggttgaaatgttcgaccaggttatcccggcagctgacaaagacatcgttaaagtcttcaccaagcgtatcagcaagaaattcaacctgatgctggaaaccaaagttaccgccgttgaagcgaaagaagacggcatttatgtgacgatggaaggcaaaaaagcacccgctgaaccgcagcgttacgacgccgtgctggtagcgattggtcgtgtgccgaacggtaaaaacctcgacgcaggcaaagcaggcgtggaagttgacgaccgtggtttcatccgcgttgacaaacagctgcgtaccaacgtaccgcacatctttgctatcggcgatatcgtcggtcaaccgatgctggcacacaaaggtgttcacgaaggtcacgttgccgctgaagttatcgccggtaagaaacactacttcgatccgaaagttatcccgtccatcgcctataccgaaccagaagttgcatgggtgggtctgactgagaaagaagcgaaagagaaaggcatcagctatgaaaccgccaccttcccgtgggctgcttctggtcgtgctatcgcttccgactgcgcagacggtatgaccaagctgattttcgacaaagaatctcaccgtgtgatcggtggtgcgattgtcggtactaacggcggcgagctgctgggtgaaatcggcctggcaatcgaaatgggttgtgatgctgaagacatcgcactgaccatccacgcgcacccgactctgcacgagtctgtgggcctggcggcagaagtgttcgaaggtagcattaccgacctgccgaacccgaaagcgaagaagaagtag

SEQ ID NO: 7 - protein, 469 aa, Corynebacterium glutamicum
MTEHYDVVVLGAGPGGYVSAIRAAQLGKKVAVIEKQYWGGVCLNVGCIPSKSLIKNAEVAHTFTHEKKTFGINGEVTFNYEDAHKRSRGVSDKIVGGVHYLMKKNKIIEIHGLGNFKDAKTLEVTDGKDAGKTITFDDCIIATGSVVNTLRGVDFSENVVSFEEQILNPVAPKKMVIVGAGAIGMEFAYVLGNYGVDVTVIEFMDRVLPNEDAEVSKVIAKAYKKMGVKLLPGHATTAVRDNGDFVEVDYQKKGSDKTETLTVDRVMVSVGFRPRVEGFGLENTGVKLTERGAIEIDDYMRTNVDGIYAIGDVTAKLQLAHVAEAQGIVAAETIAGAETQTLGDYMMMPRATFCNPQVSSFGYTEEQAKEKWPDREIKVASFPFSANGKAVGLAETDGFAKIVADAEFGELLGAHLVGANASELINELVLAQNWDLTTEEISRSVHIHPTLSEAVKEAAHGISGHMINF

SEQ ID NO: 8 - DNA, 1410 bp, Corynebacterium glutamicum
gtgactgaacattatgacgtagtagtactcggagccggccccggtggctatgtctccgccatccgtgcagcgcagcttggcaagaaggttgctgtaattgagaagcagtactggggtggtgtttgcctaaacgtgggctgcattccttccaagtctctgatcaaaaacgctgaagttgcccatacctttacccatgagaagaagaccttcggcatcaatggcgaagtgaccttcaactatgaggatgctcacaagcgttcccgtggcgtttccgacaagatcgttggaggcgttcattacttgatgaagaagaacaagatcatcgaaattcatggtcttggaaacttcaaggatgctaagactcttgaggtcaccgacggtaaggatgctggcaagaccatcacctttgatgactgcatcatcgcaaccggttcggtagtcaacaccctccgtggcgttgacttctcagagaacgttgtgtcttttgaagagcagattcttaaccctgttgcgccaaagaagatggtcattgttggtgcaggcgcaattggaatggaattcgcctacgttcttggtaactacggtgtagatgtaaccgtcatcgagttcatggatcgtgtgcttccaaatgaagatgctgaagtctccaaggttattgcaaaggcctacaagaagatgggcgttaagcttcttcctggccatgcaaccactgctgttcgggacaacggtgactttgtcgaggttgattaccagaagaagggctctgacaagacagagactcttactgttgatcgagtcatggtttccgttggtttccgtccacgcgttgagggatttggtcttgaaaacactggcgttaagctcaccgagcgtggcgcaatcgagatcgatgattacatgcgtaccaacgtcgatggcatttacgccatcggtgacgtgaccgccaagcttcagcttgctcacgtcgcagaagcacagggcattgttgccgcagagactattgctggtgcagaaactcagactcttggtgattacatgatgatgccacgtgcaaccttctgcaacccacaggtttcttcctttggttacaccgaagagcaggccaaggagaagtggccagatcgtgagatcaaggttgcttccttcccattctctgcaaacggtaaagcagttggcctggcagaaactgatggtttcgcaaagatcgttgctgatgcagaattcggtgagctgctcggtgcacacctggttggagcaaatgcatcagagctcatcaatgaattggtgcttgctcagaactgggatctcaccactgaagagatctctcgtagcgtccatattcacccaacgctatctgaggcagttaaggaagctgcacacggtatctctggacacatgatcaacttctag

SEQ ID NO: 9 - protein, 474 aa, Artificial Sequence
  Synthetic (lpdE354K)
MSTEIKTQVVVLGAGPAGYSAAFRCADLGLETVIVERYNTLGGVCLNVGCIPSKALLHVAKVIEEAKALAEHGIVFGEPKTDIDKIRTWKEKVINQLTGGLAGMAKGRKVKVVNGLGKFTGANTLEVEGENGKTVINFDNAIIAAGSRPIQLPFIPHEDPRIWDSTDALELKEVPERLLVMGGGIIGLEMGTVYHALGSQIDVVEMFDQVIPAADKDIVKVFTKRISKKFNLMLETKVTAVEAKEDGIYVTMEGKKAPAEPQRYDAVLVAIGRVPNGKNLDAGKAGVEVDDRGFIRVDKQLRTNVPHIFAIGDIVGQPMLAHKGVHEGHVAAEVIAGKKHYFDPKVIPSIAYTKPEVAWVGLTEKEAKEKGISYETATFPWAASGRAIASDCADGMTKLIFDKESHRVIGGAIVGTNGGELLGEIGLAIEMGCDAEDIALTIHAHPTLHESVGLAAEVFEGSITDLPNPKAKKK

SEQ ID NO: 10 - DNA, 1425 bp, Artificial Sequence
  Synthetic (lpdE354K)
atgagtactgaaatcaaaactcaggtcgtggtacttggggcaggccccgcaggttactccgctgccttccgttgcgctgatttaggtctggaaaccgtaatcgtagaacgttacaacacccttggcggtgtttgcctgaacgtcggctgtatcccttctaaagcactgctgcacgtagcaaaagttatcgaagaagccaaagcgctggctgaacacggtatcgtcttcggcgaaccgaaaaccgatatcgacaagattcgtacctggaaagagaaagtgatcaatcagctgaccggtggtctggctggtatggcgaaaggccgcaaagtcaaagtggtcaacggtctgggtaaattcaccggggctaacaccctggaagttgaaggtgagaacggcaaaaccgtgatcaacttcgacaacgcgatcattgcagcgggttctcgcccgatccaactgccgtttattccgcatgaagatccgcgtatctgggactccactgacgcgctggaactgaaagaagtaccagaacgcctgctggtaatgggtggcggtatcatcggtctggaaatgggcaccgtttaccacgcgctgggttcacagattgacgtggttgaaatgttcgaccaggttatcccggcagctgacaaagacatcgttaaagtcttcaccaagcgtatcagcaagaaattcaacctgatgctggaaaccaaagttaccgccgttgaagcgaaagaagacggcatttatgtgacgatggaaggcaaaaaagcacccgctgaaccgcagcgttacgacgccgtgctggtagcgattggtcgtgtgccgaacggtaaaaacctcgacgcaggcaaagcaggcgtggaagttgacgaccgtggtttcatccgcgttgacaaacagctgcgtaccaacgtaccgcacatctttgctatcggcgatatcgtcggtcaaccgatgctggcacacaaaggtgttcacgaaggtcacgttgccgctgaagttatcgccggtaagaaacactacttcgatccgaaagttatcccgtccatcgcctataccaagccagaagttgcatgggtgggtctgactgagaaagaagcgaaagagaaaggcatcagctatgaaaccgccaccttcccgtgggctgcttctggtcgtgctatcgcttccgactgcgcagacggtatgaccaagctgattttcgacaaagaatctcaccgtgtgatcggtggtgcgattgtcggtactaacggcggcgagctgctgggtgaaatcggcctggcaatcgaaatgggttgtgatgctgaagacatcgcactgaccatccacgcgcacccgactctgcacgagtctgtgggcctggcggcagaagtgttcgaaggtagcattaccgacctgccgaacccgaaagcgaagaagaagtag

SEQ ID NO: 11 - protein, 371 aa, Clostridium kluyveri
MQLFKLKSVTHHFDTFAEFAKEFCLGERDLVITNEFIYEPYMKACQLPCHFVMQEKYGQGEPSDEMMNNILADIRNIQFDRVIGIGGGTVIDISKLFVLKGLNDVLDAFDRKIPLIKEKELIIVPTTCGTGSEVTNISIAEIKSRHTKMGLADDAIVADHAIIIPELLKSLPFHFYACSAIDALIHAIESYVSPKASPYSRLFSEAAWDIILEVFKKIAEHGPEYRFEKLGEMIMASNYAGIAFGNAGVGAVHALSYPLGGNYHVPHGEANYQFFTEVFKVYQKKNPFGYIVELNWKLSKILNCQPEYVYPKLDELLGCLLTKKPLHEYGMKDEEVRGFAESVLKTQQRLLANNYVELTVDEIEGIYRRLY

SEQ ID NO: 12 - protein, 431 aa, Porphyromonas gingivalis
MKDVLAEYASRIVSAEEAVKHIKNGERVALSHAAGVPQSCVDALVQQADLFQNVEIYHMLCLGEGKYMAPEMAPHFRHITNFVGGNSRKAVEENRADFIPVFFYEVPSMIRKDILHIDVAIVQLSMPDENGYCSFGVSCDYSKPAAESAHLVIGEINRQMPYVHGDNLIHISKLDYIVMADYPIYSLAKPKIGEVEEAIGRNCAELIEDGATLQLGIGAIPDAALLFLKDKKDLGIHTEMFSDGVVELVRSGVITGKKKTLHPGKMVATFLMGSEDVYHFIDKNPDVELYPVDYVNDPRVIAQNDNMVSINSCIEIDLMGQVVSECIGSKQFSGTGGQVDYVRGAAWSKNGKSIMAIPSTAKNGTASRIVPIIAEGAAVTTLRNEVDYVVTEYGIAQLKGKSLRQRAEALIAIAHPDFREELTKHLRKRFG

SEQ ID NO: 13 - protein, 858 aa, Clostridium acetobutyricum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 14 - protein, 451 aa, Porphyromonas gingivalis
MEIKEMVSLARKAQKEYQATHNQEAVDNICRAAAKVIYENAAILAREAVDETGMGVYEHKVAKNQGKSKGVWYNLHNKKSIGILNIDERTGMIEIAKPIGVVGAVTPTTNPIVTPMSNIIFALKTCNAIIIAPHPRSKKCSAHAVRLIKEAIAPFNVPEGMVQIIEEPSIEKTQELMGAVDVVVATGGMGMVKSAYSSGKPSFGVGAGNVQVIVDSNIDFEAAAEKIITGRAFDNGIICSGEQSIIYNEADKEAVFTAFRNHGAYFCDEAEGDRARAAIFENGAIAKDVVGQSVAFIAKKANINIPEGTRILVVEARGVGAEDVICKEKMCPVMCALSYKHFEEGVEIARTNLANEGNGHTCAIHSNNQAHIILAGSELTVSRIVVNAPSATTAGGHIQNGLAVTNTLGCGSWGNNSISENFTYKHLLNISRIAPLNSSIHIPDDKEIWEL

SEQ ID NO: 15 - DNA, 170 bp, Artificial Sequence
  Synthetic (NCgl1929_promoter)
tgcgttaataaaggtggagaataagttgtttccaagatcaattcaaggaaagttgcattttcgcaggtcagtgttaccccctaagactacccctttccattgcatacaaaggaaatacatatagacttttgggcattagattacctcgataaaagtttagggaatctaaa

SEQ ID NO: 16 - DNA, 1116 bp, Clostridium kluyveri
atgcagcttttcaagctcaagagcgtcacacatcactttgatacttttgcagagtttgccaaggagttctgtctcggtgaacgcgacttggtaattaccaacgagttcatctacgaaccgtatatgaaggcatgccagctgccttgtcattttgtgatgcaggagaaatacggccaaggcgagccttctgacgagatgatgaacaacatcctagcagatatccgtaatatccagttcgaccgcgtgatcgggatcggaggtggtacggttattgacatctcaaaactctttgttctgaagggattaaatgatgttctcgacgcgttcgatcgcaagattccccttatcaaagagaaagaactgatcattgtgcccaccacctgcggaaccggctcggaggtgacgaacatttccatcgccgagatcaagtcccggcacaccaagatgggtttggctgacgatgcaattgttgctgaccacgccataatcatccctgaacttctgaagagcttgcccttccacttctatgcatgctccgcaatcgacgctcttattcatgccatcgagtcatacgtttctccaaaagcgtctccatactcccgtctgttcagtgaggcggcgtgggacattatcctggaagttttcaagaaaatcgccgaacacggcccagagtaccgcttcgagaagctgggggaaatgatcatggccagcaactatgccggtatcgctttcggcaacgcaggcgttggcgccgtccacgctctatcctacccgttgggcggcaactatcacgtgccgcatggagaagcaaactatcagttcttcaccgaggtctttaaagtataccaaaagaagaatccgttcggctatattgtcgaactcaactggaagctctccaagattctgaactgccagccagagtacgtgtacccgaagctggatgaactgctcggttgccttcttaccaagaaacctttgcacgaatacggcatgaaggacgaagaggttcgtggcttcgcggaatcggtcctgaagacccagcaacgcttgctcgccaacaactacgtcgaacttactgtcgatgagatcgaaggtatctaccgacgtctctactag

SEQ ID NO: 17 - DNA, 1296 bp, Porphyromonas gingivalis
atgaaggatgtactggcggaatacgcctcccgcattgtttcggcggaggaggccgttaagcacatcaaaaacggtgaacgggtagctttgtcacacgctgccggcgtgcctcagagttgcgttgacgcactggtgcagcaggccgaccttttccagaatgtggaaatctatcacatgctgtgcctcggtgagggtaagtatatggcgcctgagatggcccctcacttccgccacatcaccaactttgtcggtggtaactcccgtaaggcggtcgaagaaaaccgggccgatttcattccggtattcttttacgaggtgccaagcatgattcgcaaagacatcctccacattgatgtcgccatcgttcagctttcaatgcctgacgaaaatggttactgttcctttggagtatcttgcgattactccaagccggcagcagagagcgctcacctggttatcggagaaatcaaccgtcaaatgccatacgtacacggcgacaacttgattcatatctccaagttggattacatcgtgatggcagactaccccatctactctcttgcaaagcccaagatcggggaagtcgaggaagctatcgggaggaattgtgccgagcttattgaagatggtgccactctccagctgggaatcggcgcgattcctgatgcggccctgttatttctcaaggacaaaaaggatctgggcatccataccgaaatgttctccgatggtgttgtcgaattggttcgctccggcgttatcacaggcaagaaaaagactcttcaccccggaaagatggtcgcaaccttcctgatgggaagcgaggacgtgtatcatttcatcgataaaaaccccgatgtagaactgtatccagtagattacgtgaatgacccgcgtgtgatcgcccaaaacgacaatatggtctcgattaacagctgcatcgaaatcgaccttatgggacaggtcgtgtccgagtgcatcggctcaaagcaattcagcggcaccggcggccaagttgactacgtgcgtggcgcagcatggtctaaaaacggcaaatcgatcatggcaatcccgtccactgcaaaaaacggtacggcatctcgaattgtacctatcatcgcggagggcgctgctgtcaccaccctgcgcaacgaggtcgattacgttgtaaccgagtacggtatcgctcagctcaagggcaagagcctgcgccagcgcgcagaggctttgatcgcgatagcccaccccgacttccgtgaggaactaacgaaacatctccgcaagcgattcggataa

SEQ ID NO: 18 - DNA, 2577 bp, Clostridium acetobutyricum
atgaaagtaaccaatcagaaagagttgaagcagaagttgaacgagctgcgagaggctcagaagaagttcgcaacctacacccaggaacaggtggacaagatctttaagcagtgtgccattgcagccgcgaaagaacgtattaatctcgcgaaacttgcggtcgaggaaaccggtattgggctggtagaagacaagatcatcaagaaccacttcgccgctgaatacatctacaacaagtacaaaaacgaaaagacatgtggtatcatcgaccacgacgacagcttgggcatcaccaaggtagcggagccaatcggtatcgtcgcagctatcgtgcccactactaaccctacctccactgctattttcaagtcactcatctccctgaaaacccgcaatgctatcttcttctcacctcacccacgcgctaagaaatcaactatcgctgcagctaaacttatcctggatgcagccgtgaaagccggggctccgaaaaacatcatcggttggatcgacgaaccttccattgaactctctcaagacctcatgtccgaggcagacattatcctggcaaccggaggcccatccatggttaaagcagcttacagctcaggcaagccggctatcggcgttggagctggtaacactccagcaatcatcgacgagtcggccgatatcgacatggcagtgtcctctattatcctgtccaaaacttatgacaacggcgttatttgcgcgtccgagcagtctattctcgtcatgaactctatttacgagaaggtaaaggaggagtttgtgaagcgggggtcgtacattctgaaccagaacgagatcgctaagatcaaagagactatgtttaaaaacggagccatcaacgcagatatcgtagggaagtccgcgtacatcattgctaagatggctggaatcgaagtccctcaaaccacgaaaattctgatcggcgaggtgcaatcggtcgaaaagtccgagctgttctcgcatgaaaagttgtccccggtcctcgcgatgtataaagttaaggattttgatgaagcactcaagaaagctcagcgcctgatcgaattgggtggctcgggtcacacctcttccctctacattgactcccagaacaataaagataaggtgaaagagttcggcctggctatgaagacgtctcgtaccttcatcaatatgccctcttcacagggcgccagcggtgacctttacaatttcgctatcgctcctagctttaccctcggctgcggcacctggggcggtaattctgtgtcccaaaacgtcgaaccaaagcatctgctcaacattaaaagcgtcgccgaacgtcgcgagaacatgttgtggttcaaggtcccgcaaaaaatctacttcaagtatggttgcttgcgctttgcacttaaagagcttaaggacatgaataaaaagcgggcgttcatcgtcactgataaggatctgttcaaactgggctatgttaacaagattaccaaggtcctggatgagatcgatatcaagtattccatcttcaccgatattaagtccgatccgaccattgattccgtgaagaagggcgcgaaggagatgctcaactttgaacccgacacgattatttctattggcggaggcagcccaatggacgcagctaaggttatgcacctgctgtatgagtacccagaagcagagatcgagaaccttgcaatcaatttcatggatattcgcaaacgcatttgcaactttcctaagcttggtacaaaagctatctctgttgcgatccctaccaccgcaggaaccggcagcgaagcgacaccattcgccgttattaccaacgatgaaacaggtatgaagtacccacttacctcttatgaacttaccccgaacatggctatcattgatacggaattgatgctgaacatgccgcggaagttgaccgcagctacgggaatcgatgcattggttcatgcaatcgaggcatacgtttccgtcatggcaaccgattacaccgacgagctcgcgttgcgtgcgattaaaatgatcttcaagtaccttccacgcgcatacaagaatggcacaaacgatattgaagcccgagaaaagatggcacacgcttcgaacatcgctggtatggccttcgcgaatgcgtttctcggagtgtgtcactccatggcgcacaaactgggagccatgcatcacgtgccccacggtatcgcatgcgccgttcttattgaagaggtgatcaagtataatgccaccgattgccccactaagcagacggccttccctcagtacaaatcgcccaatgccaagcgtaaatacgcggaaattgccgagtacttgaaccttaaggggaccagcgacacggaaaaggtgaccgcactgattgaagccatctccaagcttaagatcgacctgagcatcccacaaaacatctcagcagccggcattaacaagaaggacttctacaacactctcgacaagatgtcagagctcgccttcgatgatcagtgcactaccgcaaacccacgttatccgctcatctctgaactgaaggatatctacatcaagtcgttttaa

SEQ ID NO: 19 - DNA, 1356 bp, Porphyromonas gingivalis
atggagattaaagagatggtcagtcttgcgcgcaaagctcagaaggagtatcaggccacccataaccaagaagctgtggacaacatctgccgagcagcagcgaaggttatttacgaaaatgcagcaattctggcacgcgaggcagtggacgaaaccggcatgggtgtttacgagcacaaggtggccaagaatcaaggcaagtccaaaggtgtttggtacaacctgcataacaagaagtcgattggcatcctcaatatcgacgagcgtaccggcatgatcgagatcgcaaaacctatcggggttgtaggcgccgttacgccaaccaccaaccctatcgttactccgatgagcaacatcatctttgctcttaagacctgcaacgccatcattatcgccccacacccgcgctccaaaaagtgctctgcccacgcagttcggctgatcaaagaggctatcgctccgttcaacgtgcccgaaggtatggttcagatcatcgaggagcctagcatcgagaagacgcaggaattgatgggcgccgtagacgtggtcgttgctaccgggggcatgggcatggtcaagtctgcctactcctcagggaagccttctttcggtgtcggagccggcaatgttcaggtgatagtggacagcaacatcgacttcgaagcggcagcagaaaagatcatcaccggacgtgccttcgacaacggtatcatctgctcaggcgaacagtccatcatctacaacgaggctgacaaggaagcagttttcacagcattccgcaaccacggtgcgtacttttgcgacgaggccgagggagatcgggctcgtgcagcgatcttcgaaaatggagccatcgcgaaagatgttgtgggccagtccgttgcctttattgcaaagaaggcgaacattaatatccccgagggtactcgtattctcgtggtcgaagctcgcggagtaggcgccgaagatgtcatctgtaaagaaaagatgtgtccagtcatgtgcgccctctcctacaagcacttcgaagagggggtagagatcgcaaggacgaacctcgcaaacgaaggcaatggccatacctgtgctatccactccaacaaccaagcacacatcatcttggcaggctcggagctgaccgtgtctcgcatcgtggtcaacgcgccaagtgctaccacagcaggcggtcacatccagaacggtcttgccgtcaccaatactctaggctgcggctcttggggtaacaactcgatctccgaaaacttcacttataaacacctgctcaacatttcacgcatcgccccgttgaactccagcattcatatcccagatgataaggaaatctgggaactctaa

SEQ ID NO: 20 - protein, 538 aa, Clostridium kluyveri
MSKGIKNSQLKKKNVKASNVAEKIEEKVEKTDKVVEKAAEVTEKRIRNLKLQEKVVTADVAADMIENGMIVAISGFTPSGYPKEVPKALTKKVNALEEEFKVTLYTGSSTGADIDGEWAKAGIIERRIPYQTNSDMRKKINDGSIKYADMHLSHMAQYINYSVIPKVDIAIIEAVAITEEGDIIPSTGIGNTATFVENADKVIVEINEAQPLELEGMADIYTLKNPPRREPIPIVNAGNRIGTTYVTCGSEKICAIVMTNTQDKTRPLTEVSPVSQAISDNLIGFLNKEVEEGKLPKNLLPIQSGVGSVANAVLAGLCESNFKNLSCYTEVIQDSMLKLIKCGKADVVSGTSISPSPEMLPEFIKDINFFREKIVLRPQEISNNPEIARRIGVISINTALEVDIYGNVNSTHVMGSKMMNGIGGSGDFARNAYLTIFTTESIAKKGDISSIVPMVSHVDHTEHDVMVIVTEQGVADLRGLSPREKAVAIIENCVHPDYKDMLMEYFEEACKSSGGNTPHNLEKALSWHTKFIKTGSMK

SEQ ID NO: 21 - DNA, 1617 bp, Clostridium kluyveri
atgtctaaaggaatcaagaatagccaattgaaaaaaaagaacgtcaaggccagtaacgttgctgagaagatcgaagagaaggtggaaaagaccgacaaggtcgttgagaaggctgctgaggtgaccgaaaagcgcattcgaaacttaaagctccaggaaaaagttgtgaccgcagatgtcgcagctgacatgatcgagaatggcatgatcgtcgcaattagcggcttcacgccatccgggtatccaaaggaggttccaaaagcccttactaagaaggttaatgcgctggaggaggagttcaaggtgacgctgtataccggttctagcacaggcgctgatattgacggagaatgggcgaaggcaggaataatcgaacggcgtatcccataccagaccaactctgacatgaggaaaaaaataaacgatggttcaatcaagtacgcagatatgcacctgagccacatggctcaatacattaactattctgtgattcctaaggttgacattgccatcatcgaggcggtggccattaccgaggaaggggatattattcctagtactggaatcggcaacacagctacgtttgtcgagaatgcggataaggtaattgtggaaataaacgaggctcagccgcttgagttggaaggcatggcagatatctataccctgaagaaccctccacgtcgcgagcccatcccgatagtcaacgcaggcaaccgcatagggaccacttacgtcacctgtggctctgaaaaaatctgcgcgatcgtcatgaccaacacccaagacaaaacccgcccactcaccgaagtttctcctgtcagtcaggcaatctccgataacctgattggcttcctgaacaaagaagtagaggagggtaaactcccaaaaaacctgctccccatacagtcaggtgtcggttcggttgctaacgccgttctagccggactctgcgaatcaaacttcaaaaatttgagctgctacacagaagtgatccaggattcgatgttgaagctcatcaaatgtggaaaggcagatgtggtgtccggcacctcgatctcgccatcaccggaaatgctgcccgagttcataaaggacataaatttttttcgcgagaagatagtactgcgcccccaggaaatatctaataatccggaaatagctcgtcgtataggagtgatctccataaacactgctttggaagtagacatctacggtaatgtgaactccacgcatgtcatgggctccaagatgatgaacggcatcggcggcagcggcgactttgcccgcaacgcatacctcaccatattcactacggagtccatcgcgaagaagggcgacatttcctctatcgttcctatggtttcccacgtggaccacaccgagcatgacgtaatggtcatcgttaccgaacagggggttgcggatctgcgcggtctttcccctcgggaaaaggccgtggcgataattgagaattgcgtccacccggattacaaggatatgctcatggagtacttcgaggaggcttgtaagtcctcaggtggcaacaccccacacaaccttgaaaaagccctatcctggcacactaagttcataaaaactggctcgatgaagtaa

SEQ ID NO: 22 - DNA, 43 bp, Artificial Sequence
  Synthetic (ldhA_5′_HindIII)
catgattacgccaagcttgagagcccaccacattgcgatttcc

SEQ ID NO: 23 - DNA, 42 bp, Artificial Sequence
  Synthetic (ldhA_up_3′_XhoI)
tcgaaactcgagtttcgatcccacttcctgatttccctaacc

SEQ ID NO: 24 - DNA, 39 bp, Artificial Sequence
  Synthetic (ldhA_dn_5′_XhoI)
tcgaaactcgagtaaatctttggcgcctagttggcgacg

SEQ ID NO: 25 - DNA, 46 bp, Artificial Sequence
  Synthetic (ldhA_3′_EcoRI)
acgacggccagtgaattcgacgacatctgagggtggataaagtggg

SEQ ID NO: 26 - DNA, 20 bp, Artificial Sequence
  Synthetic (ldhA up)
atcgggcataattaaaggtg

SEQ ID NO: 27 - DNA, 22 bp, Artificial Sequence
  Synthetic (ldhA down)
gtcacctcatcaagttctagaa

SEQ ID NO: 28 - DNA, 6702 bp, Artificial Sequence
  Synthetic (4gene_cat1_sucD_4hbd_cat2)
tctagaatgactattaatgtctccgaactacttgccaaagtccccacgggtctactgattggtgattcctgggtggaagcatccgacggcggtactttcgatgtggaaaacccagcgacgggtgaaacaatcgcaacgctcgcgtctgctacttccgaggatgcactggctgctcttgatgctgcatgcgctgttcaggccgagtgggctaggatgccagcgcgcgagcgttctaatattttacgccgcggttttgagctcgtagcagaacgtgcagaagagttcgccaccctcatgaccttggaaatgggcaagcctttggctgaagctcgcggcgaagtcacctacggcaacgaattcctgcgctggttctctgaggaagcagttcgtctgtatggccgttacggaaccacaccagaaggcaacttgcggatgctgaccgccctcaagccagttggcccgtgcctcctgatcaccccatggaacttcccactagcaatggctactagatgattttgcatctgctgcgaaatctttgtttccccgctaaagttgaggacaggttgacacggagttgactcgacgaattatccaatgtgagtaggtttggtgcgtgagttggaaaaattcgccatactcgcccttgggttctgtcagctcaagaattcttgagtgaccgatgctctgattgacctaactgcttgacacattgcatttcctacaatctttagaggagacacaacatgtctaaaggaatcaagaatagccaattgaaaaaaaagaacgtcaaggccagtaacgttgctgagaagatcgaagagaaggtggaaaagaccgacaaggtcgttgagaaggctgctgaggtgaccgaaaagcgaattcgaaacttaaagctccaggaaaaagttgtgaccgcagatgtcgcagctgacatgatcgagaatggcatgatcgtcgcaattagcggcttcacgccatccgggtatccaaaggaggttccaaaagcccttactaagaaggttaatgcgctggaggaggagttcaaggtgacgctgtataccggttctagcacaggcgctgatattgacggagaatgggcgaaggcaggaataatcgaacggcgtatcccataccagaccaactctgacatgaggaaaaaaataaacgatggttcaatcaagtacgcagatatgcacctgagccacatggctcaatacattaactattctgtgattcctaaggttgacattgccatcatcgaggcggtggccattaccgaggaaggggatattattcctagtactggaatcggcaacacagctacgtttgtcgagaatgcggataaggtaattgtggaaataaacgaggctcagccgcttgagttggaaggcatggcagatatctataccctgaagaaccctccacgtcgcgagcccatcccgatagtcaacgcaggcaaccgcatagggaccacttacgtcacctgtggctctgaaaaaatctgcgcgatcgtcatgaccaacacccaagacaaaacccgcccactcaccgaagtttctcctgtcagtcaggcaatctccgataacctgattggcttcctgaacaaagaagtagaggagggtaaactcccaaaaaacctgctccccatacagtcaggtgtcggttcggttgctaacgccgtgcatcccggactctgcgaatcaaacttcaaaaatttgagctgctacacagaagtgatccaggattcgatgttgaagctgatcaaatgtggaaaggcagatgtggtgtccggcacctcgatctcgccatcaccggaaatgctgcccgagttcataaaggacataaatttttttcgcgagaagatagtactgcgcccccaggaaatatctaataatccggaaatagctcgtcgtataggagtgatctccataaacactgctttggaagtagacatctacggtaatgtgaactccacgcatgtcatgggctccaagatgatgaacggcatcggcggcagcggcgactttgcccgcaacgcatacctcaccatattcactacggagtccatcgcgaagaagggcgacatttcctctatcgttcctatggtttcccacgtggaccacaccgagcatgacgtaatggtcatcgttaccgaacagggggttgcggatctccgcggtctttcccctcgggaaaaggccgtggcgataattgagaattgcgtccacccggattacaaggatatgctcatggagtacttcgaggaggcttgtaagtcctcaggtggcaacaccccacacaaccttgaaaaagccctatcctggcacactaagttcataaaaactggctcgatgaagtaattagaggagacacaacatggagattaaagagatggtcagtcttgcgcgcaaagctcagaaggagtatcaggccacccataaccaagaagctgtggacaacatctgccgagctgcagcgaaggttatttacgaaaatgcagcaattctggcccgcgaggcagtggacgaaaccggcatgggtgtttacgagcacaaggtggccaagaatcaaggcaagtccaaaggtgtttggtacaacctgcataacaagaagtcgattggcatcctcaatatcgatgagcgtaccggcatgatcgagatcgcaaaacctatcggggttgtaggcgccgttacgccaaccaccaaccctatcgttactccgatgagcaacatcatctttgctcttaagacctgcaacgccatcattatcgccccacacccgcgctccaaaaagtgctctgcccacgcagttcggctgatcaaagaggctatcgctccgttcaacgtgcccgaaggtatggttcagatcatcgaggagcctagcatcgagaagacgcaggaattgatgggcgccgtagacgtggtcgttgctaccgggggcatgggcatggtcaagtctgcctactcctcagggaagccttctttcggtgtcggagccggcaatgttcaggtgatagtggacagcaacatcgatttcgaagcggctgcagaaaagatcatcaccggacgtgccttcgacaacggtatcatctgctcaggcgaacagtccatcatctacaacgaggctgacaaggaagcagttttcacagcattccgcaaccacggtgcgtacttttgcgacgaggccgagggagatcgggctcgtgcagcgatcttcgaaaatggagccatcgcgaaagatgttgtgggccagtccgttgcctttattgccaagaaggcgaacattaatatccccgagggtactcgtattctcgtggtcgaagctcgcggagtaggcgccgaagatgtcatctgtaaagaaaagatgtgtccagtcatgtgcgccctctcctacaagcacttcgaagagggggtagagatcgcaaggacgaacctcgcaaacgaaggcaatggccatacctgtgctatccactccaacaaccaagcacacatcatcttggcaggctcggagctgaccgtgtctcgcatcgtggtcaacgcgccaagtgctaccacagcaggcggtcacatccagaacggtcttgccgtcaccaatactctaggctgcggctcttggggtaacaactcgatctccgaaaacttcacttataaacacctgctcaacatttcacgcatcgccccgttgaactccagcattcatatcccagatgataaggaaatctgggaactctaattagaggagacacaacatgcagcttttcaagctcaagagcgtcacacatcactttgatacttttgcagagtttgccaaggaattctgtctcggtgaacgcgacttggtaattaccaacgagttcatctacgaaccgtatatgaaggcatgccagctgccttgtcattttgtgatgcaggagaaatacggccaaggcgagccttctgacgagatgatgaacaacatcctagcagatatccgtaatatccagttcgaccgcgtgatcgggatcggaggtggtacggttattgacatctcaaaactctttgttctgaagggattaaatgatgttctcgacgcgttcgatcgcaagattccccttatcaaagagaaagaactgatcattgtgcccaccacctgcggaaccggctcggaggtgacgaacatttccatcgccgagatcaagtcccggcacaccaagatgggtttggctgacgatgcaattgttgctgaccacgccataatcatccctgaacttctgaagagcttgcccttccacttctatgcatgctccgcaatcgatgctcttattcatgccatcgagtcatacgtttctccaaaagcgtctccatactcccgtctgttcagtgaggcggcgtgggacattatcctggaagttttcaagaaaatcgccgaacacggcccagagtaccgcttcgagaagctgggggaaatgatcatggccagcaactatgccggtatcgctttcggcaacgcaggcgttggcgccgtccacgctctatcctacccgttgggcggcaactatcacgtgccgcatggagaagcaaactatcagttcttcaccgaggtctttaaagtataccaaaagaagaatccgttcggctatattgtcgaactcaactggaagctctccaagattctgaactgccagccagagtacgtgtacccgaagctggatgaactgctcggttgccttcttaccaagaaacctttgcacgaatacggcatgaaggacgaagaggttcgtggcttcgcggaatcggtcctgaagacccagcaacgcttgctcgccaacaactacgtcgaacttactgtcgatgagatcgaaggtatctaccgacgtctctactaattagaggagacacaacatgaaggatgtactggcggaatacgcctcccgcattgtttcggcggaggaggccgttaagcacatcaaaaacggtgaacgggtagctttgtcacacgctgccggcgtgcctcagagttgcgttgacgcactggtgcagcaggccgaccttttccagaatgtggaaatctatcacatgctgtgcctcggtgagggtaagtatatggcgcctgagatggcccctcacttccgccacatcaccaactttgtcggtggtaactcccgtaaggcggtcgaagaaaaccgggccgatttcattccggtattcttttacgaggtgccaagcatgattcgcaaagacatcctccacattgatgtcgccatcgttcagctttcaatgcctgacgaaaatggttactgttcctttggagtatcttgcgattactccaagccggcagcagagagcgctcacctggttatcggagaaatcaaccgtcaaatgccatacgtacacggcgacaacttgattcatatctccaagttggattacatcgtgatggcagactaccccatctactctcttgcaaagcccaagatcggggaagtcgaggaagctatcgggaggaattgtgccgagcttattgaagatggtgccactctccagctgggaatcggcgcgattcctgatgcggccctgttatttctcaaggacaaaaaggatctgggcatccataccgaaatgttctccgatggtgttgtcgaattggttcgctccggcgttatcacaggcaagaaaaagactcttcaccccggaaagatggtcgcaaccttcctgatgggaagcgaggacgtgtatcatttcatcgataaaaaccccgatgtagaactgtatccagtagattacgtgaatgacccgcgtgtgatcgcccaaaacgacaatatggtctcgattaacagctgcatcgaaatcgaccttatgggacaggtcgtgtccgagtgcatcggctcaaagcaattcagcggcaccggcggccaagttgactacgtgcgtggcgcagcatggtctaaaaacggcaaatcgatcatggcaatcccgtccactgcaaaaaacggtacggcatctcgaattgtacctatcatcgcggagggcgctgctgtcaccaccctgcgcaacgaggtcgattacgttgtaaccgagtacggtatcgctcagctcaagggcaagagcctgcgccagcgcgcagaggctttgatcgcgatagcccaccccgacttccgtgaggaactaacgaaacatctccgcaagcgattcggataacatatggcggccgcaagcttgcctcgacgaaggcgtcaccgtgggccccctggttgaggaaaaagcacgagacagcgttgcatcgcttgtcgacgccgccgtcgccgaaggtgccaccgtcctcaccggcggcaaggccggcacaggtgcaggctacttctacgaaccaacggtgctcacgggagtttcaacagatgcggctatcctgaacgaagagatcttcggtcccgtcgcaccgatcgtcaccttccaaaccgaggaagaagccctgcgtctagccaactccaccgaatacggactggcctcctatgtgttcacccaggacacctcacgtattttccgcgtctccgatggtctcgagttcggcctagtgggcgtcaattccggtgtcatctctaacgctgctgcaccttttggtggcgtaaaacaatccggaatgggccgcgaaggtggtctcgaaggaatcgaggagtacacctccgtgcagtacatcggtatccgggatccttacgccggctaggctagc

SEQ ID NO: 29 - DNA, 36 bp, Artificial Sequence
  Synthetic (0049-1 for)
gcaggcatgcaagcttaaagtccccacgggtctact

SEQ ID NO: 30 - DNA, 36 bp, Artificial Sequence
  Synthetic (0049-2 rev)
ggccagtgccaagctttaccgatgtactgcacggag

SEQ ID NO: 31 - DNA, 3608 bp, Artificial Sequence
  Synthetic (adhE2_nt)
aagcttgcatgcctgcaggtcgactctagaggatccccgggaggcacctcacaggtgcaattattacacaaccccacagcgatgtccgcatcctttgatgaccccaacctcatctcgcttgctggactggttccaaccatgcacttagccgatgctgccagcctgtccaccttggcccaggaccggttgagcatcaccggtgataaaggtgccaatgctggtgcgaagatcgcctccctagtcgcgggcatggtcgccggtgctgattccatcgatgacatggatgtactccgccacggaggtatgcgccgacttttcgaccggatctacgccccatccacattggggtcttttctgcgggccttcactttcggccacgtacgccaactcgatgattttgcatctgctgcgaaatctttgtttccccgctaaagttgaggacaggttgacacggagttgactcgacgaattatccaatgtgagtaggtttggtgcgtgagttggaaaaattcgccatactcgcccttgggttctgtcagctcaagaattcttgagtgaccgatgctctgattgacctaactgcttgacacattgcatttcctacaatctttagaggagacacaacatgaaagtaaccaatcagaaagagttgaagcagaagttgaacgagctgcgagaggctcagaagaagttcgcaacctacacccaggaacaggtggacaagatctttaagcagtgtgccattgcagccgcgaaagaacgtattaatctcgcgaaacttgcggtcgaggaaaccggtattgggctggtagaagacaagatcatcaagaaccacttcgccgctgaatacatctacaacaagtacaaaaacgaaaagacatgtggtatcatcgaccacgacgacagcttgggcatcaccaaggtagcggagccaatcggtatcgtcgcagctatcgtgcccactactaaccctacctccactgctattttcaagtcactcatctccctgaaaacccgcaatgctatcttcttctcacctcacccacgcgctaagaaatcaactatcgctgcagctaaacttatcctggatgcagccgtgaaagccggggctccgaaaaacatcatcggttggatcgacgaaccttccattgaactctctcaagacctcatgtccgaggcagacattatcctggcaaccggaggcccatccatggttaaagcagcttacagctcaggcaagccggctatcggcgttggagctggtaacactccagcaatcatcgacgagtcggccgatatcgacatggcagtgtcctctattatcctgtccaaaacttatgacaacggcgttatttgcgcgtccgagcagtctattctcgtcatgaactctatttacgagaaggtaaaggaggagtttgtgaagcgggggtcgtacattctgaaccagaacgagatcgctaagatcaaagagactatgtttaaaaacggagccatcaacgcagatatcgtagggaagtccgcgtacatcattgctaagatggctggaatcgaagtccctcaaaccacgaaaattctgatcggcgaggtgcaatcggtcgaaaagtccgagctgttctcgcatgaaaagttgtccccggtcctcgcgatgtataaagttaaggattttgatgaagcactcaagaaagctcagcgcctgatcgaattgggtggctcgggtcacacctcttccctctacattgactcccagaacaataaagataaggtgaaagagttcggcctggctatgaagacgtctcgtaccttcatcaatatgccctcttcacagggcgccagcggtgacctttacaatttcgctatcgctcctagctttaccctcggctgcggcacctggggcggtaattctgtgtcccaaaacgtcgaaccaaagcatctgctcaacattaaaagcgtcgccgaacgtcgcgagaacatgttgtggttcaaggtcccgcaaaaaatctacttcaagtatggttgcttgcgctttgcacttaaagagcttaaggacatgaataaaaagcgggcgttcatcgtcactgataaggatctgttcaaactgggctatgttaacaagattaccaaggtcctggatgagatcgatatcaagtattccatcttcaccgatattaagtccgatccgaccattgattccgtgaagaagggcgcgaaggagatgctcaactttgaacccgacacgattatttctattggcggaggcagcccaatggacgcagctaaggttatgcacctgctgtatgagtacccagaagcagagatcgagaaccttgcaatcaatttcatggatattcgcaaacgcatttgcaactttcctaagcttggtacaaaagctatctctgttgcgatccctaccaccgcaggaaccggcagcgaagcgacaccattcgccgttattaccaacgatgaaacaggtatgaagtacccacttacctcttatgaacttaccccgaacatggctatcattgatacggaattgatgctgaacatgccgcggaagttgaccgcagctacgggaatcgatgcattggttcatgcaatcgaggcatacgtttccgtcatggcaaccgattacaccgacgagctcgcgttgcgtgcgattaaaatgatcttcaagtaccttccacgcgcatacaagaatggcacaaacgatattgaagcccgagaaaagatggcacacgcttcgaacatcgctggtatggccttcgcgaatgcgtttctcggagtgtgtcactccatggcgcacaaactgggagccatgcatcacgtgccccacggtatcgcatgcgccgttcttattgaagaggtgatcaagtataatgccaccgattgccccactaagcagacggccttccctcagtacaaatcgcccaatgccaagcgtaaatacgcggaaattgccgagtacttgaaccttaaggggaccagcgacacggaaaaggtgaccgcactgattgaagccatctccaagcttaagatcgacctgagcatcccacaaaacatctcagcagccggcattaacaagaaggacttctacaacactctcgacaagatgtcagagctcgccttcgatgatcagtgcactaccgcaaacccacgttatccgctcatctctgaactgaaggatatctacatcaagtcgttttaatttgatcacggccattcaccaccgtaaccggtagctccctgaccacccagccgagctttcggcgtgagatgacaacaattcgtggaacaaccagaacaagacgtgatctggcgatcacccctacccgaaaattccggacccgcccggaaccgggatcaggacatcaccgagggcacatcggtggatcgaggcttaatggaacgccccactcatccaatccggcaattttgatgctgtacccatcgacgcatggtgctccaaatacgtggaagccatcacggtcacggatgaagcatggcaggttttccggttggaagtccactggattgttgggcaggaaccaggtgagcgcctgaatggcgaatggcgataagctagaggatccccgggtaccgagctcgaattc

SEQ ID NO: 32 - DNA, 20 bp, Artificial Sequence
  Synthetic (AdhE2_1_F)
atgaaagtaaccaatcagaa

SEQ ID NO: 33 - DNA, 20 bp, Artificial Sequence
  Synthetic (AdhE2_2260_R)
aatcggtggcattatacttg

SEQ ID NO: 34 - DNA, 21 bp, Artificial Sequence
  Synthetic (MD-404)
cccaggctttacactttatgc

SEQ ID NO: 35 - DNA, 40 bp, Artificial Sequence
  Synthetic (MD-615)
gcgtaatagcgaagaggggcgtttttccataggctccgcc

SEQ ID NO: 36 - DNA, 46 bp, Artificial Sequence
  Synthetic (MD-616)
aaagtgtaaagcctgggaacaacaagacccatcatagtttgccccc

SEQ ID NO: 37 - DNA, 40 bp, Artificial Sequence
  Synthetic (MD-617)
gttcaatcataacaccccttgtattactgtttatgtaagc

SEQ ID NO: 38 - DNA, 36 bp, Artificial Sequence
  Synthetic (MD-618)
gttcttctaatcagaattggttaattggttgtaaca

SEQ ID NO: 39 - DNA, 31 bp, Artificial Sequence
  Synthetic (MD-619)
gggtgttatgattgaacaagatggattgcac

SEQ ID NO: 40 - DNA, 39 bp, Artificial Sequence
  Synthetic (MD-620)
attctgattagaagaactcgtcaagaaggcgatagaagg

SEQ ID NO: 41 - DNA, 17 bp, Artificial Sequence
  Synthetic (LacZa-NR)
cctcttcgctattacgc

SEQ ID NO: 42 - DNA, 48 bp, Artificial Sequence
  Synthetic (MD-625)
tagggcgaattgggtaccatgattttgcatctgctgcgaaatctttgt

SEQ ID NO: 43 - DNA, 55 bp, Artificial Sequence
  Synthetic (MD-626)
gataccgtcgacctcgaggttgtgtctcctctaaagattgtaggaaatgcaatgt

SEQ ID NO: 44 - DNA, 47 bp, Artificial Sequence
  Synthetic (MD-627)
gccaccgcggtggagctcatttagcggatgattctcgttcaacttcg

SEQ ID NO: 45 - DNA, 32 bp, Artificial Sequence
  Synthetic (MD-628)
ttttatttgcaaaaacggccgaaaccatccct

SEQ ID NO: 46 - DNA, 40 bp, Artificial Sequence
  Synthetic (MD-629)
ccgtttttgcaaataaaacgaaaggctcagtcgaaagact

SEQ ID NO: 47 - DNA, 43 bp, Artificial Sequence
  Synthetic (MD-630)
gaacaaaagctggagctaccgtatctgtggggggatggcttgt

SEQ ID NO: 48 - DNA, 50 bp, Artificial Sequence
  Synthetic (J0180)
ctatagggcgaattgggtacctgcgttaataaaggtggagaataagttgt

SEQ ID NO: 49 - DNA, 41 bp, Artificial Sequence
  Synthetic (MD-1081)
tgacctcctctcgagtttagattccctaaacttttatcgag

SEQ ID NO: 50 - DNA, 36 bp, Artificial Sequence
  Synthetic (MD-1082)
aaactcgagaggaggtcatgatgagtactgaaatca

SEQ ID NO: 51 - DNA, 36 bp, Artificial Sequence
  Synthetic (MD-1083)
ttattcctcctacttcttcttcgctttcgggttcgg

SEQ ID NO: 52 - DNA, 37 bp, Artificial Sequence
  Synthetic (MD-1084)
aagaagtaggaggaataacccatgtcagaacgtttcc

SEQ ID NO: 53 - DNA, 22 bp, Artificial Sequence
  Synthetic (MD-1085)
ttttacctcctacgccagacgc

SEQ ID NO: 54 - DNA, 26 bp, Artificial Sequence
  Synthetic (MD-1086)
tctggcgtaggaggtaaaagaataat

SEQ ID NO: 55 - DNA, 43 bp, Artificial Sequence
  Synthetic (MD-1087)
ggtggcggccgctctagattacatcaccagacggcgaatgtca

SEQ ID NO: 56 - DNA, 27 bp, Artificial Sequence
  Synthetic (MD-1088)
cctataccaagccagaagttgcatggg

SEQ ID NO: 57 - DNA, 26 bp, Artificial Sequence
  Synthetic (MD-1089)
acttctggcttggtataggcgatgga

(19) United States
(12) Patent Application Publication
(10) Pub. No.: US 2015/0064758 A1
(43) Pub. Date: Mar. 5, 2015

(54) MICROORGANISM COMPRISING PYRUVATE DEHYDROGENASE VARIANT AND METHOD OF PRODUCING C4-CHEMICALS USING THE SAME

(71) Applicant: Samsung Electronics Co., Ltd., Suwon-si (KR)

(72) Inventors: Wooyong Lee, Hwaseongi-si (KR); Joonsong Park, Seoul (KR); Youngmin Lee, Suwon-si (KR); Jaechan Park, Yongin-si (KR); Jinhwan Park, Suwon-si (KR)

(21) Appl. No.: 14/472,596

(22) Filed: Aug. 29, 2014

(30) Foreign Application Priority Data
Aug. 29, 2013 (KR) 10-2013-0103427

(51) Int. Cl. C12N 15/77; C12P 7/18

(52) U.S. Cl. CPC C12N 15/77; C12P 7/18
USPC 435/158; 435/252.32; 435/471

(57) ABSTRACT

A recombinant microorganism including pyruvate dehydrogenase having increased activity may increase 1,4-BDO production under anaerobic conditions, as well as a method for preparing same, and method of using same to produce a C4 chemical.

### CROSS-REFERENCE TO RELATED APPLICATIONS

This application claims the benefit of Korean Patent Application No. 10-2013-0103427, filed on Aug. 29, 2013, in the Korean Intellectual Property Office, the disclosure of which is hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: 92,218 bytes ASCII (Text) file named “718240_ST25.TXT,” created Aug. 28, 2014.

### BACKGROUND

1. Field

The present disclosure relates to methods of activating a tricarboxylic acid (TCA) cycle of an aerobic strain or a Corynebacterium strain under anaerobic conditions. In addition, the present disclosure relates to a microorganism of which a TCA cycle is active under anaerobic conditions and a method of efficiently producing C4-chemicals using the same.

2. Description of the Related Art

1,4-butanediol (1,4-BDO) is used not only as a solvent for manufacturing plastics and fiber but also as a raw material for producing fibers such as spandex. About 1.3 million tons of 1,4-BDO is produced in a year worldwide from petroleum-based materials such as acetylene, butane, propylene, and butadiene. In addition, a 6% increase in consumption is anticipated each year.

1,4-butanediol is important as it is used throughout the entire chemical industry for the production of various chemicals such as polymers, solvents, and fine chemical intermediates. Most chemicals containing four carbons are currently synthesized by being derived from 1,4-butanediol or maleic anhydride, but the chemical production process needs to be improved or replaced by a newly developed process as production costs are increasing due to rising oil prices. Thus, biological processes using microorganisms are suggested as alternative processes.

Microorganisms of Corynebacterium genus are gram positive strains and used in industries for producing amino acids such as glutamate, lysine, threonine, and isoleucine. Growth conditions of microorganisms of Corynebacterium genus are simple, and the microorganisms allow for high growth. In addition, mutation rarely takes place in the microorganisms because the genome structure is stable. Moreover, microorganisms of Corynebacterium genus are non-pathogenic and harmless to the environment as they do not produce a spore. Microorganisms of Corynebacterium genus are aerobic bacteria; under anaerobic conditions where oxygen supply is prevented or insufficient, metabolic processes of a Corynebacterium bacterium are stopped except for a metabolic process of producing the minimum energy for survival. In addition, microorganisms of Corynebacterium genus produce lactic acid, acetic acid or succinic acid for energy production.

To produce 1,4-BDO, most microorganisms including a Corynebacterium bacterium should be cultured under anaerobic conditions. However, biological metabolic pathways in a microorganism do not efficiently operate under anaerobic conditions. Therefore, 1,4-BDO is not effectively produced under anaerobic conditions as energy and metabolic intermediates necessary to produce 1,4-BDO and other products are insufficient. Thus, there remains a need for genetically modified microorganisms that efficiently produce 1,4-BDO under anaerobic conditions.

### SUMMARY

An aspect provides a genetically modified microorganism comprising a polynucleotide encoding a pyruvate dehydrogenase that remains active or has increased activity compared to an unmodified microorganism of the same type under anaerobic conditions, as well as a method of preparing the microorganism.

Another aspect provides a method of producing 1,4-BDO using the microorganism.

### BRIEF DESCRIPTION OF THE DRAWINGS

These and/or other aspects will become apparent and more readily appreciated from the following description of the embodiments, taken in conjunction with the accompanying drawings of which:

FIG. 1 is a flowchart depicting pyruvate and a variety of metabolites used in the TCA cycle, and enzymes necessary in the TCA cycle;

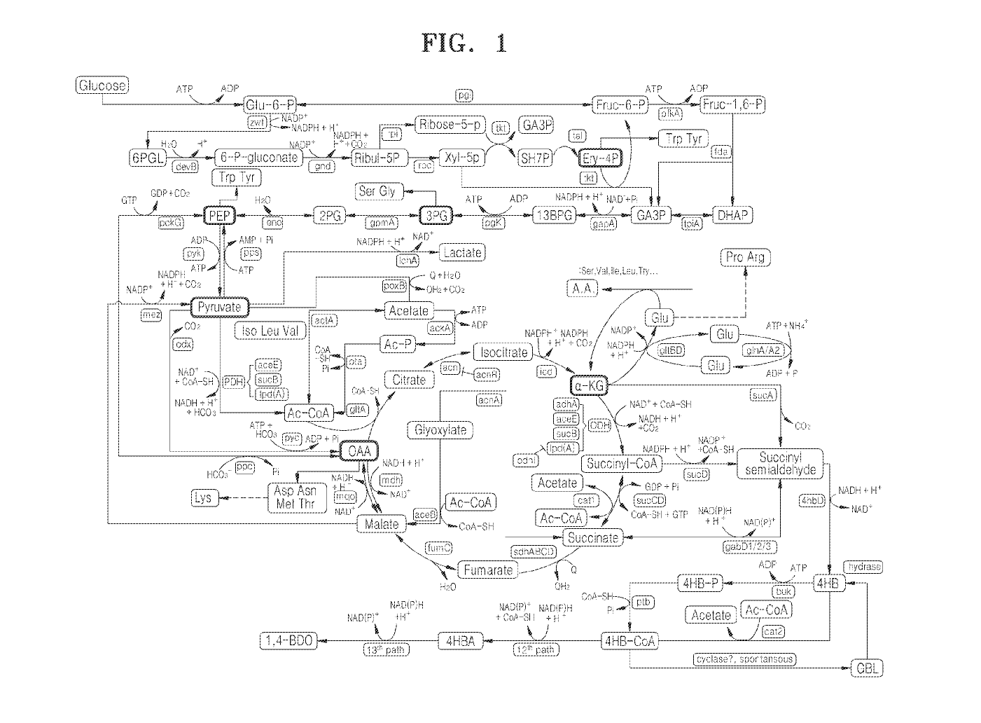

FIG. 2 is a graph displaying a comparison of the PDH activity of a Corynebacterium strain (ATCC13032 Δldh) under aerobic conditions and under anaerobic conditions;

FIG. 3A is a vector map of pGS-Term which is a Corynebacteria exogenous expression vector.

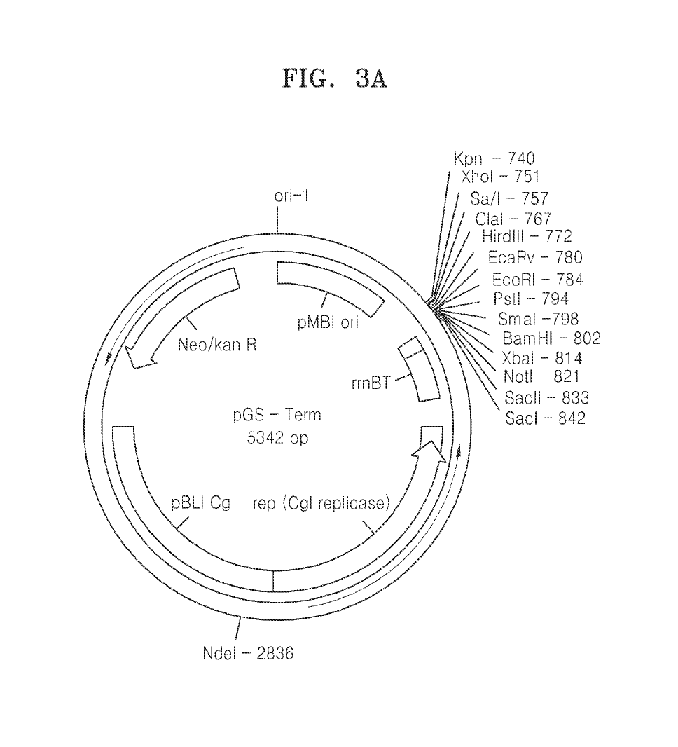

FIG. 3B is a vector map of MD0375 which is a control vector.

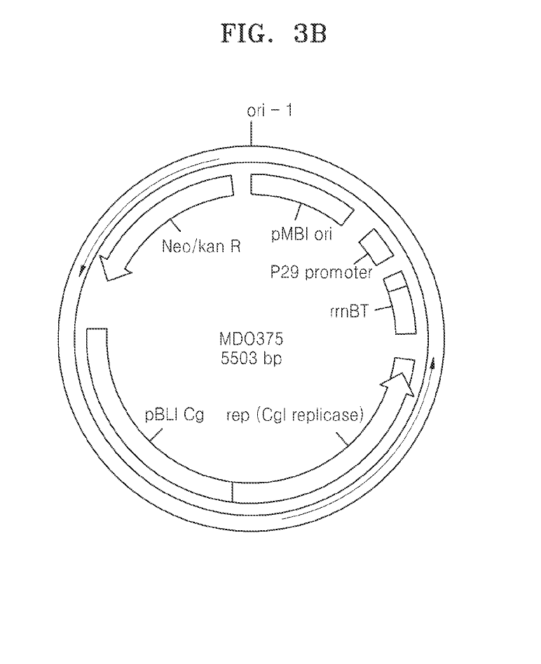

FIG. 3C is a vector map of MD0376 for expressing an E. coli PDH complex.

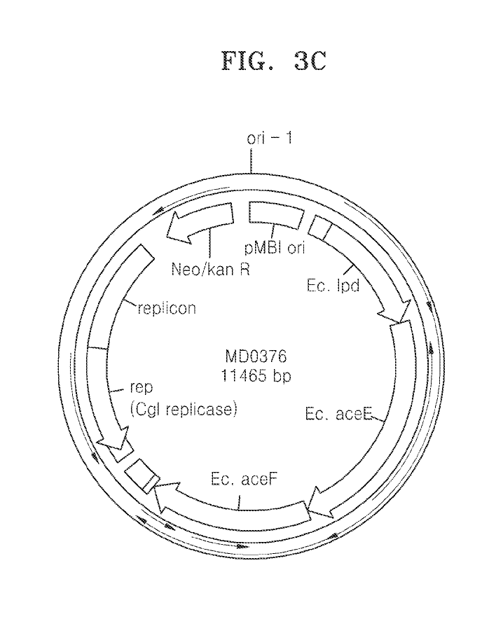

FIG. 3D is a vector map of MD0377 for expressing a mutant of an E. coli PDH complex including lpd^{E354K} mutant. lpd^{E354K} represents that the mutant is formed by substituting Glu-354 with lysine;

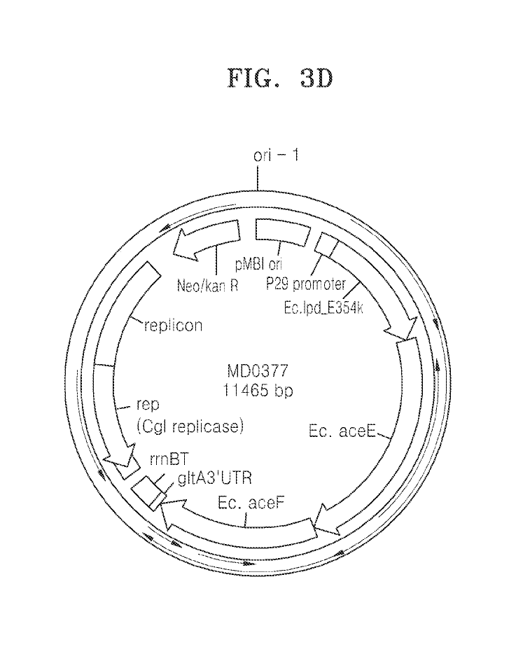

FIG. 4 is a graph displaying a comparison of the PDH activity of a Corynebacterium strain prepared by using the vector in FIG. 3 under aerobic conditions and under anaerobic conditions; and

FIG. 5 is a graph displaying the PDH activity of a Corynebacterium strain wherein NCgI0355 (lpd) gene or NCgI0658 (lpdA) gene is eliminated under aerobic conditions and under anaerobic conditions.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

An aspect provides a genetically modified microorganism comprising a polynucleotide encoding a pyruvate dehydrogenase (PDH) that remains active or has increased activity under anaerobic conditions, compared to an unmodified microorganism of the same type. The term “unmodified microorganism of the same type” refers to a reference microorganism with that does not comprise a modification of interest (i.e., a subject modification). The reference microorganism refers to a wild-type microorganism or a parental microorganism. The parental microorganism refers to a microorganism that has not undergone a subject modification but is genetically identical to the genetically modified microorganism except for the modification, and thus serves as a reference microorganism for the modification.

The term “anaerobic conditions” herein refers to a state in which oxygen content is lower than that of a normal atmospheric state. The anaerobic conditions may represent a state in which oxygen content is lower than 21% in the air at the site where culturing is performed. In addition, the oxygen content under anaerobic conditions may be lower than that of the atmosphere, as the oxygen content may be, for example, lower than 20%, 15%, 10%, 5% or 1%. In addition, dissolved oxygen in a culture medium under anaerobic conditions may be lower than 10 ppm, 8 ppm, 5 ppm, 3 ppm or 2 ppm or the dissolved oxygen may be in a range from about 0.1 ppm to about 1 ppm.

Pyruvate dehydrogenase refers to a “pyruvate dehydrogenase complex.” Pyruvate dehydrogenase is an enzyme having an activity of catalyzing conversion of pyruvate to acetyl-CoA. Pyruvate dehydrogenase includes pyruvate dehydrogenase (E1), dihydrolipoyl transacetylase (E2), and dihydrolipoyl dehydrogenase (E3). In the pyruvate dehydrogenase, E1 is also referred to as AceE, E2 is referred to as AceF, and E3 is referred to as Lpd or LpdA, depending on microorganisms.

The pyruvate dehydrogenase (E1) catalyzes a reaction in which pyruvate is converted to acetyl-CoA by combining pyruvate and thiamine pyrophosphate (TPP). The pyruvate dehydrogenase (E1) may be an enzyme classified as EC 1.2.4.1. The dihydrolipoyl transacetylase (E2) has an activity of catalyzing transacylation. The dihydrolipoyl transacetylase (E2) may be an enzyme classified as EC 2.3.1.12. The dihydrolipoyl dehydrogenase (E3) catalyzes conversion of FAD to FADH₂, through a reaction in which flavin-mediated oxidation, and conversion of pyruvate to acetyl-CoA occurs. The dihydrolipoyl dehydrogenase (E3) may be an enzyme classified as EC 1.8.1.4.

The genetically modified microorganism may comprise a polynucleotide encoding a pyruvate dehydrogenase of which activity under anaerobic conditions is more than 90%, 80%, 70%, 60%, 50%, 40% or 30% of the activity present under aerobic conditions. In contrast, the unmodified microorganism of the same type comprises a polynucleotide encoding a pyruvate dehydrogenase of which activity under anaerobic conditions is less than 22% of activity under aerobic condition. Aerobic conditions represent a state that is the same as or similar to the normal atmospheric state or a state in which dissolved oxygen is the same as that of normal atmospheric state.

The pyruvate dehydrogenase that remains active or has increased activity under anaerobic conditions may be a mutant of the pyruvate dehydrogenase included in an Escherichia coli. The pyruvate dehydrogenase may include AceE protein or a mutant thereof, AceF protein or a mutant thereof, and Lpd protein or a mutant thereof. The AceE protein is referred to as pyruvate dehydrogenase subunit E1 or pyruvate dehydrogenase (E1). The AceE protein may include an amino acid sequence of SEQ ID NO: 1. The AceF protein is referred to as pyruvate dehydrogenase subunit E2 or dihydrolipoyl acetyltransferase (E2). The AceF protein may include an amino acid sequence of SEQ ID NO: 3. The Lpd protein is referred to as pyruvate dehydrogenase subunit E3 or dihydrolipoyl dehydrogenase (E3). The Lpd protein may include an amino acid sequence of SEQ ID NO: 5. The mutant of the Lpd protein may include lysine instead of glutamic acid corresponding to the 354th amino acid in SEQ ID NO: 5 (Refer to SEQ ID NO: 9). The pyruvate dehydrogenase may include pyruvate dehydrogenase (E1)) protein, dihydrolipoyl transacetylase (E2) protein, and a mutant of dihydrolipoyl dehydrogenase (E3) protein.

The genetically modified microorganism may produce 1,4-BDO. The unmodified microorganism of the same type may also be a microorganism capable of producing 1,4-BDO. In addition, the genetically modified microorganism may be a microorganism which has become capable of producing 1,4-BDO after genes related to 1,4-BDO biosynthesis have been introduced. The microorganism may be a microorganism of Corynebacterium genus. The microorganism of Corynebacterium genus may be Corynebacterium glutamicum.

The microorganism capable of producing 1,4-BDO may include an enzyme that catalyzes the conversion of succinyl CoA to succinyl semialdehyde, an enzyme that catalyzes the conversion of succinyl semialdehyde to 4-hydroxybutyrate, an enzyme that catalyzes the conversion of 4-hydroxybutyrate to 4-hydroxybutyrate-CoA, and an enzyme that catalyzes the conversion of 4-hydroxybutyrate-CoA to 1,4-BDO.

The enzyme that catalyzes the conversion of succinyl CoA to succinyl semialdehyde may be CoA-dependent succinate semialdehyde dehydrogenase. The enzyme may be an enzyme classified as EC 1.2.1. An example of the enzyme may be SucD. The enzyme that catalyzes the conversion of succinyl semialdehyde to 4-hydroxybutyrate may be 4-hydroxybutyrate (4HB) dehydrogenase. The enzyme may be an enzyme classified as EC 1.1.1. The enzyme may be 4Hbd. In addition, the enzyme that catalyzes the conversion of 4-hydroxybutyrate to 4-hydroxybutyrate-CoA may be 4-hydroxybutyryl CoA transferase. The enzyme may be an enzyme classified as EC 2.8.3. An example of the enzyme may be Cat2. The enzyme that catalyzes the conversion of 4-hydroxybutyrate-CoA to 1,4-BDO may be alcohol dehydrogenase. The alcohol dehydrogenase may be AdhE or AdhE2. The AdhE2 may be an enzyme classified as EC.1.1.1. As an example, the microorganism producing 1,4-BDO may be a microorganism expressing the SucD protein, the 4Hbd protein, the Cat2 protein, and the AdhE2 protein.

The term “protein expression” herein means that a protein or an enzyme exists (i.e., is produced in) and has activity in a microorganism. A polynucleotide which encodes a protein is transcribed to an mRNA which is in turn translated into the protein. The polynucleotide encoding the protein may exist either by being inserted in a chromosome of a microorganism or by being inserted in a plasmid vector.

The CoA-dependent succinate semialdehyde dehydrogenase may be a protein derived from an Escherichia genus, a Corynebacterium genus or a Porphyromonas genus. In an embodiment of the present invention, the SucD protein may have an amino acid sequence of SEQ ID NO: 14. The polynucleotide encoding the SucD may have a nucleotide sequence of SEQ ID NO: 19.

The 4HB dehydrogenase may be a protein derived from an Escherichia genus, a Corynebacterium genus or a Porphyromonas genus. In an embodiment of the present invention, the 4Hbd protein may have an amino acid sequence of SEQ ID NO: 11. The polynucleotide encoding the 4Hbd may have a nucleotide sequence of SEQ ID NO: 16.

The 4-hydroxybutyryl CoA transferase may be a protein derived from an Escherichia genus, a Corynebacterium genus or a Porphyromonas genus. The 4-hydroxybutyryl CoA transferase is also referred to as Cat2. In an embodiment of the present invention, the Cat2 protein may have an amino acid sequence of SEQ ID NO: 12. The polynucleotide encoding the Cat2 may have a nucleotide sequence of SEQ ID NO: 17.

The alcohol dehydrogenase may be a protein derived from Clostridium acetobutylicum. The AdhE2 protein may have an amino acid sequence of SEQ ID NO: 13. The polynucleotide encoding the AdhE2 may have a nucleotide sequence of SEQ ID NO: 18.

The microorganism may additionally include succinyl CoA:coenzyme A transferase. The succinyl CoA:coenzyme A transferase of the microorganism may have an activity to catalyze a reaction converting succinate to succinyl CoA. The succinyl CoA:coenzyme A transferase is also referred to as Cat1. In an embodiment of the present invention, Cat1 may be an enzyme classified as EC.2.8.3. The Cat1 may have an amino acid sequence of SEQ ID NO: 20. The polynucleotide encoding the Cat1 may have a nucleotide sequence of SEQ ID NO: 21.

The genetically modified microorganism may be a microorganism in which a pathway for synthesizing lactate from pyruvate is inactivated or decreased. The pathway synthesizing lactate from pyruvate may be catalyzed by lactate dehydrogenase. Lactate dehydrogenase is an enzyme that catalyzes the conversion of pyruvate to lactate. The lactate dehydrogenase (LDH) may include lactate dehydrogenase A (LdhA), lactate dehydrogenase B (LdhB), and lactate dehydrogenase C (LdhC). The activity of the lactate dehydrogenase may be eliminated or decreased in the genetically modified microorganism. The lactate dehydrogenase may be an enzyme classified as EC.1.1.1.27. The genetically modified microorganism may be a microorganism wherein a gene encoding lactate dehydrogenase is inactivated or attenuated.

The term “inactivation” herein may mean that a gene is not expressed or a gene is expressed but a product of the expressed gene is not active. The term “attenuation” may mean that expression of a gene is decreased to a level lower than an expression level of wild type strain, a strain which is not genetically engineered or a parent strain, or that a gene is expressed but a product of the expressed gene has a decreased activity. A decreased Ldh activity in the microorganism may be lower than 30%, 20% or 10% of the Ldh activity of wild type microorganism. The microorganism may be formed by completely eliminating the Ldh activity. The inactivation or the attenuation may be caused by homologous recombination. The inactivation or attenuation may be performed by transforming a vector including a part of the sequence of the genes into a cell, culturing the cell so that homologous recombination of the sequence may occur with an endogenous gene of the cell, and then selecting a cell in which homologous recombination has occurred using a selection marker. The term “decrease” may represent relatively lowered activity of the genetically engineered microorganism in comparison to activity of a microorganism which is not genetically engineered.

Activity of the lactate dehydrogenase may be inactivated or attenuated in the microorganism by a mutation of gene encoding the lactate dehydrogenase. The mutation may be performed by substitution, partial or total deletion or addition of a nucleotide. Activity of the lactate dehydrogenase in the microorganism may be decreased by eliminating an endogenous lactate dehydrogenase gene. The elimination includes not only physical elimination of the gene but also prevention of functional expression of the gene. The elimination may be performed by homologous recombination.

The term “transformation” herein refers to introducing a gene into a microorganism so that the gene may be expressed in the microorganism. If the gene may be expressed in the microorganism, may be inserted into a chromosome of the microorganism or may exist outside a chromosome or in a plasmid vector. The gene may be DNA or RNA. The introduction of the gene may be any type of introduction, so long as the gene may be introduced into and expressed in the microorganism. For example, the gene may be introduced into a microorganism by an introduction in the form of an expression cassette, which is a polynucleotide structure including all factors related to the expression of the gene by itself. The expression cassette usually includes a promoter, a transcription termination signal, a ribosome binding site, and a translation termination signals operably linked with the gene. The expression cassette may be an expression vector capable of self-replication. The gene may be introduced as itself or in the form of a polynucleotide structure to a host cell and then be operably linked with a sequence related to an expression in the microorganism.

Another aspect provides a method of preparing a genetically modified microorganism that produces 1,4-BDO under anaerobic conditions including introduction of a polynucleotide encoding a pyruvate dehydrogenase that remains active or has increase activity under anaerobic conditions compared to an unmodified microorganism of the same type into a microorganism; inactivation or attenuation of lactate dehydrogenase activity; and introduction of a polynucleotide encoding 4HB dehydrogenase, a polynucleotide encoding 4-hydroxybutyryl CoA transferase, a polynucleotide encoding alcohol dehydrogenase, a polynucleotide encoding CoA-dependent succinate semialdehyde dehydrogenase, and a polynucleotide encoding SucA.

The method of preparing the genetically modified microorganism is specifically described below.

The polynucleotide encoding the pyruvate dehydrogenase that remains active or has increase activity under anaerobic conditions may include pyruvate dehydrogenase (E1) protein (AceE), dihydrolipoyl transacetylase (E2) protein (AceF), and a mutant of dihydrolipoyl dehydrogenase (E3) protein (Lpd). The polynucleotide encoding the AceE protein may have a nucleotide sequence of SEQ ID NO: 2. The polynucleotide encoding the AceF protein may have a nucleotide sequence of SEQ ID NO: 4. The polynucleotide encoding the Lpd protein may have a nucleotide sequence of SEQ ID NO: 6 or SEQ ID NO: 8. The polynucleotide encoding the mutant of the Lpd protein may have a nucleotide sequence of SEQ ID NO: 10.

The polynucleotide may be introduced to a microorganism through a vector. The term “vector” refers to a DNA product including a DNA sequence operably linked with an appropriate regulation sequence capable of expressing DNA in an appropriate host cell. The vector may be a plasmid vector, a bacteriophage vector, and a cosmid vector.

To operate as an expression vector, a vector may include a replication origin, a promoter, a multi-cloning site (MCS), a selection marker or a combination thereof. A replication origin gives a function to a plasmid to replicate itself independently of a host cell chromosome. A promoter operates in transcription process of an inserted foreign gene. An MCS enables a foreign gene to be inserted through various restriction enzyme sites. A selection marker verifies whether a vector has been properly introduced to a host cell. A selection includes an antibiotic-resistant gene generally used in the art. For example, a selection marker may include a gene resistant to ampicillin, gentamycin, carbenicillin, chloramphenicol, streptomycin, kanamycin, geneticin, neomycin or tetracycline. Considering the cost, an ampicillin or gentamycin-resistant gene may be used.

When a vector of an aspect uses a prokaryotic cell as the host cell, a strong promoter, for example, a lamda-PL promoter, a trp promoter, a lac promoter or a T7 promoter, is included in the vector. If a vector uses a eukaryotic cell as the host cell, the vector may include a promoter derived from a genome of a mammal (a metallothionin promoter, e.g.) or a promoter derived from a mammal virus (an adenovirus late promoter, a vaccinia virus 7.5K promoter, a SV40 promoter, cytomegalovirus promoter or a tk promoter of a HSV promoter, e.g.). The promoter may be a lambda-PL promoter, a trp promoter, a lac promoter or a T7 promoter. In this manner, a promoter is operably linked with a sequence encoding a gene.

The term “operably linked” herein may mean a functional bond between a nucleic acid expression regulatory sequence (promoter, signal sequence or array at transcription regulation factor binding site) and another nucleic acid sequence. Through the functional bond, the regulatory sequence may control transcription and/or translation of a nucleic acid encoding the gene.

In addition, the microorganism may be formed by eliminating or decreasing activity of lactate dehydrogenase. Activity of lactate dehydrogenase may be repressed by substitution, partial or total deletion, or addition of bases of the gene encoding lactate dehydrogenase. Activity of lactate dehydrogenase may be repressed by substituting the lactate dehydrogenase gene with a gene without lactate dehydrogenase activity. The lactate dehydrogenase may be L-lactate dehydrogenase.

In addition, according to an embodiment of the present invention, the microorganism may include 4Hbd protein, Cat2 protein, AdhE2 protein or SucD protein. The 4Hbd protein may have an amino acid sequence of SEQ ID NO: 11. The Cat2 protein may have an amino acid sequence of SEQ ID NO: 12. The AdhE2 protein may have an amino acid sequence of SEQ ID NO: 13. The SucD protein may have an amino acid sequence of SEQ ID NO: 14.

According to an embodiment of the present invention, the method may include introduction of a polynucleotide encoding 4Hbd, a polynucleotide encoding Cat2, a polynucleotide encoding AdhE2, and a polynucleotide encoding SucD to the microorganism. The microorganism may include a polynucleotide encoding 4Hbd protein, Cat2 protein, AdhE2 protein or SucD protein. In addition, the proteins may exist in the microorganism as the polynucleotides are expressed in the microorganism. The polynucleotides may be introduced to the microorganism through a vector. In addition, the polynucleotide encoding 4Hbd may have a nucleotide sequence of SEQ ID NO: 16. The polynucleotide encoding Cat2 may have a nucleotide sequence of SEQ ID NO: 17. The polynucleotide encoding AdhE2 may have a nucleotide sequence of SEQ ID NO: 18. The polynucleotide encoding SucD may have a nucleotide sequence of SEQ ID NO: 19.

Another aspect provides a method of producing C4-chemicals using a genetically modified microorganism under anaerobic conditions. The genetically modified microorganism is described above. In addition, the genetically modified microorganism may be a microorganism prepared by the preparation method described above.

The culturing may be performed according an appropriate culture medium and culture conditions known in the art. The culture medium and culture conditions may be conveniently adjusted according to the selected microorganism. The culturing method may include batch culturing, continuous culturing, fed-batch culturing or a combination thereof.

The culture medium may include various carbon sources, nitrogen sources, and trace elements.

The carbon source may include a carbohydrate such as glucose, sucrose, lactose, fructose, maltose, starch, and cellulose, a lipid such as soybean oil, sunflower oil, castor oil, and coconut oil, a fatty acid such as palmitic acid, stearic acid, and linoleic acid, an organic acid such as acetic acid or a combination thereof. The culturing may be performed by using glucose as a carbon source. The nitrogen source may include an organic nitrogen source such as peptone, yeast extract, meat extract, malt extract, corn steep liquid, and soybean, an inorganic nitrogen source such as urea, ammonium sulfate, ammonium chloride, ammonium phosphate, ammonium carbonate, and ammonium nitrate or a combination thereof. The culture medium may include as a phosphorous source, for example, potassium dihydrogen phosphate, dipotassium phosphate, a sodium-containing salt corresponding to potassium dihydrogen phosphate, and dipotassium phosphate, and a metal salt such as magnesium sulfate and iron sulfate. The culture medium or an individual component may be added to the culture in a batch mode or a continuous mode.

The culture medium or an individual component may be added to the culture solution in a batch mode or a continuous mode.

In addition, pH of the culture may be adjusted during the culturing by adding a compound such as ammonium hydroxide, potassium hydroxide, ammonia, phosphoric acid or sulfuric acid to the culture in an appropriate mode. In addition, bubble formation may be repressed by using an endoplasmic reticulum such as fatty acid polyglycol ester.

The microorganism may be cultured under anaerobic conditions. The term “anaerobic conditions” herein refers to a state in which oxygen content is lower than that of a normal atmospheric state. Anaerobic conditions may be formed, for example, by supplying carbon dioxide or nitrogen at a flow rate range from about 0.1 vvm (Volume per Volume per Minute) to about 0.4 vvm, from about 0.2 vvm to about 0.3 vvm, or at a flow rate of 0.25 vvm. In addition, anaerobic conditions may be formed by setting an aeration rate in a range from about 0 vvm and to 0.4 vvm, from about 0.1 vvm to about 0.3 vvm, or from 0.15 vvm to about 0.25 vvm.

The method of producing C4-chemicals includes recovering the produced C4 organic chemicals from the culture. The produced C4-chemicals may be succinate, fumaric acid, malic acid or a C4-chemical derived therefrom. According to one embodiment of the present invention, the produced C4-chemicals may be 4-HB, 1,4-BDO, GBL or a C4-chemical derived therefrom. For example, the recovery of 4-HB may be performed by using known separation and purification methods. The recovery may be performed by centrifugation, ion exchange chromatography, filtration, precipitation or a combination thereof.

The method of producing C4-chemicals may be used to yield various organic compounds by converting C4-chemicals to other organic chemicals. A substrate structurally related to 4-HB may be synthesized by chemically converting the 4-HB yielded in the method described above. According to one embodiment of the present invention, gamma butyrolactone (GBL) may be yielded by reacting 4-HB at about 100° C. to 200° C. in the presence of a strong acid and then distilling the reactant. The yielded GBL may be converted to N-methyl pyrrolidone (NMP) by amination using an aminating agent, for example, methylamine. In addition, the yielded GBL may be selectively converted to tetrahydrofuran (THF), 1,4-BDO or butanol by hydrogenation using a metal-containing catalyst, for example, Ru or Pd.

The poly-4-hydroxybutirate may be yielded by biologically converting the produced 4-HB. The biological conversion may be by polyhydroxyalkanoate synthase, 4-HB-CoA:coenzyme A transferase or a combination thereof.

As described above, the microorganism according to the one embodiment maintains a TCA cycle even under anaerobic conditions. In addition, the microorganism is capable of producing chemicals using metabolic intermediates of the TCA cycle even under anaerobic conditions. Various C4-chemicals may be produced using the metabolic intermediates of the TCA cycle by maintaining the TCA cycle under anaerobic conditions. Thus, the production efficiency of industrially useful products such as 1,4-BDO may be increased by using the microorganism. Therefore, the microorganism and the method of producing C4-chemicals using the same according to an embodiment have high industrial applicability.

Metabolites are not well produced in vivo under anaerobic conditions with insufficient oxygen. To resolve insufficiency of acety-CoA, one of the metabolites, a PDH enzyme maintained under anaerobic conditions, was developed. A microorganism including the enzyme obtained thereby may efficiently produce fermentation products. Therefore, such a transformed microorganism may have high industrial applicability.

It should be understood that the following examples described herein should be considered in a descriptive sense only and not for purposes of limitation. Descriptions of features or aspects within each embodiment should typically be considered as available for other similar features or aspects in other embodiments.

### Example 1

### Preparation of Corynebacterium Microorganism in which Endogenous Lactate Dehydrogenase Gene is Deleted

A decrease in intracellular acetyl-CoA concentration was found to occur when culturing Corynebacterium glutamicum ATCC13032 under anaerobic conditions. Therefore, it was assumed that a decrease in TCA cycle activity may be caused by the decrease in the acetyl-CoA concentration. In addition, an experiment was designed to search for a method to resolve the problem. For this, a Δldh Corynebacterium microorganism ATCC13032 in which an endogenous lactate dehydrogenase gene is deleted (hereinafter referred to as “basic strain”) was prepared by deleting the endogenous lactate dehydrogenase gene in Corynebacterium glutamicum, which is the natural Corynebacterium glutamicum, so that the PDH enzyme activity might be conveniently measured.

### 1.1 Preparation of Replacement Vector

The L-lactate dehydrogenase gene of Corynebacterium glutamicum (CGL) ATCC13032 was inactivated by homologous recombination using a pK19 mobsacB (ATCC87098) vector.

The two homologous regions for the elimination of the ldh gene were obtained by PCR amplification using the genome DNA of CGL ATCC13032. Two homologous regions for the elimination of the ldhA gene were located upstream and downstream from the gene and obtained by PCR amplification using a primer set including ldhA_{—}5′_HindIII (SEQ ID NO: 22) and ldhA_up_{—}3′_XhoI (SEQ ID NO: 23) and a primer set including ldhA_dn_{—}5′_XhoI (SEQ ID NO: 24) and ldhA_{—}3′_EcoRI (SEQ ID NO: 25). The PCR amplification was performed by repeating, 30 times, a cycle including a denaturation step at 95° C. for 30 seconds, an annealing step at 55° C. for 30 seconds, and an extension step at 72° C. for 30 seconds. All the PCR amplifications hereinafter were performed under the same conditions.

A pK19_Δldh vector was prepared by cloning the obtained amplification product to the HindIII and EcoRI restrict enzyme positions of a pK19 mobsacB vector.

1.2 Preparation of CGL (ΔldhA) strain

The pK19_Δldh vector was introduced into CGL ATCC13032 by electroporation. The strain in which the vector was introduced was cultured at 30° C. by streaking the strain on a lactobacillus selection (LBHIS) culture medium including kanamycin 25 μg/ml. The LBHIS culture medium includes brain-heart infusion broth 18.5 g/L, 0.5 M sorbitol, 5 g/L bacto-tryptone, 2.5 g/L bacto-yeast extract, 5 g/L NaCl, and 18 g/L bacto-agar. Hereinafter, composition of the LBHIS medium is the same. The colony was streaked on a LB-sucrose culture medium and cultured at 30° C. Then, only the colonies in which double crossing over occurred were selected. The genome DNA was separated from the selected colonies, and deletion of the ldh gene was verified through PCR by using primer sets ldhA up (SEQ ID NO: 26) and ldhA down (SEQ ID NO: 27). The CGL (ΔldhA) strain was obtained as a result.

### Example 2

### Introduction of Genes for 1,4-BDO Production

A CGL strain capable of producing 1,4-BDO was prepared on the basis of the strain prepared above. To insert four genes of cat1, sucD, 4hbD, and cat2 into a chromosome of the strain, a pK19 gapA::4G vector for the insertion of cat1, sucD 4hbD, and cat2 genes was prepared on the basis of pK19 mobsacB. The pK19 gapA::4G vector was prepared by synthesizing a whole 4G gene having a nucleotide sequence of SEQ ID NO: 28 and cloning the 4G gene into the NheI and XbaI restriction enzyme sites of the pK19 mobsacB vector.

2.1 Preparation of CGL (ΔldhA 4G) Strain

The pK19 gapA::4G vector was introduced into CGL (Δldh) by electroporation. The strain in which the pK19 gapA::4G vector was introduced was cultured at 30° C. by streaking the strain on a LBHIS culture medium including kanamycin 25 μg/ml. The colony was streaked on a LB-sucrose culture medium and cultured at 30° C. Then, only the colonies in which double crossing-over occurred were selected. The genome DNA was separated from the selected colonies, and introduction of the 4G genes was verified through PCR by using primer sets 0049-1 for (SEQ ID NO: 29) and 0049-2 rev (SEQ ID NO: 30). The CGL (Δldh 4G) strain was obtained as a result.

### Example 3

### Preparation of Strain in which adhE2 is Introduced

3.1 Preparation of pK19 gapA::adhE2 Vector

To insert the adhE2 gene into the chromosome, the pK19 gapA::adhE2 vector for insertion of the adhE2 gene was prepared on the basis of pK19 mobsacB. The pK19 gapA::adhE2 was prepared by synthesizing a whole adhE2 gene having a nucleotide sequence of SEQ ID NO: 31 and cloning the adhE2 gene into the SmaI restriction enzyme site of the pK19 mobsacB vector.

3.2 Preparation of CGL (Δldh 4G adhE2) Strain

The pK19 gapA::adhE2 vector was introduced into CGL (Δldh 4G) by electroporation. The strain in which the pK19 gapA::adhE2 vector was introduced was cultured at 30° C. by streaking the strain on LBHIS culture medium including kanamycin 25 μg/ml. The colony was streaked on LB-sucrose culture medium and cultured at 30° C. Then, only the colonies in which double crossing over occurred were selected. The genome DNA was separated from the selected colonies, and introduction of the adhE2 gene was verified through PCR by using primer sets AdhE2_{—}1_F for (SEQ ID NO: 32) and AdhE2_{—}2260_R (SEQ ID NO: 33). The CGL (Δldh 4G adhE2) strain capable of producing 1,4-BDO was obtained as a result.

### Example 4

### Preparation of Strain Wherein PDH Activity is Increased Under Anaerobic Conditions

### 4.1 Preparation of Vector

(1) Preparation of pGS-Term

The following four PCR products were obtained by using Phusion High-Fidelity DNA Polymerase (New England Biolabs, cat. # M0530). PCR was performed by using the CGL promoter screening vector pET2 as a template with the primer sequences MD-616 (SEQ ID NO: 36) and MD-618 (SEQ ID NO: 38), and with the primer sequences MD-615 (SEQ ID NO: 35) and MD-617 (SEQ ID NO: 37). PCR was performed by using a mammalian fluorescence protein expression vector pEGFP-C1 (Clonetech) as a template with the primer sequences MD-619 (SEQ ID NO: 39) and MD-620 (SEQ ID NO: 40). PCR was performed by using a E. coli cloning vector pBluescriptII SK+ as a template with primer sequences LacZa-NR (SEQ ID NO: 41) and MD-404 (SEQ ID NO: 34). The respective PCR products, 3010 bp, 854 bp, 809 bp, and 385 bp were cloned to a circular plasmid by using a In-Fusion EcoDry PCR Cloning Kit (Clonetech, cat. #639690) method.

The cloned vector including the 3010 bp, 854 bp, 809 bp, and 385 bp PCR products above was transformed to a One Shot TOP10 Chemically Competent Cell (Invitrogen, cat. # C4040-06), which was then cultured in a LB culture medium including kanamycin 25 mg/L. Growing colonies were selected and the vector was recovered from selected colonies. Then, the vector sequences were verified through total sequence analysis. The vector was named as pGluscriptII SK+. To prepare a CGL shuttle vector including a transcription terminator and a 3′ untranslated region (UTR), a 3′UTR of CGL gltA (NCgI0795) and a rho-independent terminator of rrnB of E. coli rrnB were inserted to the pGluscriptII SK+vector. A 108 bp PCR fragment of gltA 3′UTR was obtained by performing PCR using CGL (ATCC13032) genome DNA as a template with the primer sequences MD-627 (SEQ ID NO: 44) and MD-628 (SEQ ID NO: 45).

In addition, an rrnB transcription terminator 292 bp PCR product was obtained by performing PCR using E. coli (MG1655) genome DNA as a template with the primer sequences MD-629 (SEQ ID NO: 46) and MD-630 (SEQ ID NO: 47). The two amplified fragments were inserted into SacI digested pGSK+ by using an In-Fusion EcoDry PCR Cloning Kit (Clonetech, cat. #639690). The cloned vector including the two amplified fragments was transformed to a One Shot TOP10 Chemically Competent Cell (Invitrogen, cat. # C4040-06), which was then cultured in a LB culture medium including kanamycin 25 mg/L. Growing colonies were selected, and the vector was recovered from selected colonies. Then, the vector sequences were verified through total sequence analysis. The vector was named as pGS-Term.

### (2) Preparation of MD0375

A 206 bp PCR product was obtained by amplifying CGL NCgI1929 promoter through PCR using J0180 (SEQ ID NO: 48) and MD-1081 (SEQ ID NO: 49) primers. The 206 bp PCR product was inserted into a pGS-Term vector cleaved by KpnI/XhoI. The cloned vector including the 206 bp PCR product was transformed to a One Shot TOP10 Chemically Competent Cell (Invitrogen, cat. # C4040-06), which was then cultured in a LB culture medium including kanamycin 25 mg/L. The vector was recovered from the colonies. Then, the vector sequences were verified through total sequence analysis. The vector was named as MD0375.

### (3) Preparation of MD0376

A CGL shuttle vector wherein each gene of E. coli PDH complex is over-expressed under NCgI1929 promoter was prepared. 206 bp, 1454 bp, 2694 bp, and 1935 bp DNA fragments were obtained by performing PCR using CGL NCgI1929 promoter, Ec.lpd open reading frame (SEQ ID NO: 5) encoding E. coli dehydrolipoamide dehydrogenase next to natural ribosome binding site, Ec.aceE open reading frame (SEQ ID NO: 1) encoding E. coli pyruvate dehydrogenase next to natural ribosome binding site, and Ec.aceF open reading frame (SEQ ID NO: 3) encoding E. coli dihydrolipoamide acetyltransferase next to natural ribosome binding site, with primers J0180 (SEQ ID NO: 48) and MD-1081 (SEQ ID NO: 49), MD-1082 (SEQ ID NO: 50) and MD-1083 (SEQ ID NO: 51), MD-1084 (SEQ ID NO: 52) and MD-1085 (SEQ ID NO: 53), and MD-1086 (SEQ ID NO: 54) and MD-1087 (SEQ ID NO: 55), respectively.

The DNA fragments were ligated with KpnI/XbaI digested pGS-Term vector using In-Fusion EcoDry PCR Cloning Kit (Clonetech, cat. #639690). The cloned vector including the 206 bp, 1454 bp, 2694 bp, and 1935 bp DNA fragments was transformed to a One Shot TOP10 Chemically Competent Cell (Invitrogen, cat. # C4040-06), which was then cultured in a LB culture medium including kanamycin 25 mg/L. The vector was recovered from the colonies. Then, the vector preparation was verified through total sequence analysis. The vector was named as MD0376.

### (4) Preparation of MD0377

206 bp, 2694 bp, and 1935 bp DNA fragments were obtained by performing PCR using CGL NCgI1929 promoter, Ec.aceE open reading frame (SEQ ID NO: 1) encoding E. coli pyruvate dehydrogenase next to natural ribosome binding site, and Ec.aceF open reading frame (SEQ ID NO: 3) encoding E. coli dihydrolipoamide acetyltransferase next to natural ribosome binding site, with primers MD-1082 (SEQ ID NO: 50) and MD-1083 (SEQ ID NO: 51), MD-1084 (SEQ ID NO: 52) and MD-1085 (SEQ ID NO: 53), and MD-1086 (SEQ ID NO: 54) and MD-1087 (SEQ ID NO: 55), respectively. To clone a NADH-insensitive point mutation formed by substituting Glu-354 of E. coli dehydrolipoamide dehydrogenase with lysine, PCR was performed by using Ec.lpd open reading frame with the primer sequences MD-1082 (SEQ ID NO: 50) and MD-1089 (SEQ ID NO: 57), and with MD-1083 (SEQ ID NO: 51) and MD-1088 (SEQ ID NO: 56), and two overlapped fragments of 1090 bp and 383 bp were obtained, respectively.

The respective fragments were ligated with KpnI/XbaI digested pGS-Term vector using In-Fusion EcoDry PCR Cloning Kit (Clonetech, cat. #639690). The cloned vector was transformed to a One Shot TOP10 Chemically Competent Cell (Invitrogen, cat. # C4040-06), which was then cultured in a LB culture medium including kanamycin 25 mg/L. The vector was recovered from the colonies. Then, the vector preparation was verified through total sequence analysis. The vector was named as MD0377.

### 4.2 Preparation of Strains

The pGS-Term, MD0375, MD0376, and MD0377 vectors were respectively transformed into the CGL (Δldh 4G adhE2) strain by the method of Example 1. A growing colony was streaked on a LB-sucrose culture medium and cultured at 30° C. Then, only colonies in which double crossing over occurred were selected. The genome DNA was separated from the selected colonies, and C. glutamicum strains in which pGS-Term, MD0375, MD0376, or MD0377 vector was introduced were obtained. Next, the obtained C. glutamicum was cultured in 10 mL of a LBHIS culture medium contained in a 125 mL flask at 30° C. for 16 hours. To verify whether the prepared vector was included in the C. glutamicum cells, cells were taken from 3 mL of the culture solution including the LBHIS culture medium and treated with 250 uL 1×TE buffer including lysozyme 6 mg/mL for three hours. Existence and size of the vector were verified by agarose gel electrophoresis through a general mini-prep method.

The culture for cell growth was performed under aerobic conditions, and the cells were cultured under aerobic conditions and anaerobic conditions to measure PDH activity. Aerobic conditions were maintained by stirring the flask at a rate of 230 rpm under general atmospheric conditions, while anaerobic conditions were maintained by injecting nitrogen into the flask.

### Example 5

### Investigation of Cause for TCA Cycle Decrease Under Anaerobic Conditions

To verify the relationship between anaerobic conditions and low acetyl-CoA concentration, the PDH enzyme activity was measured in the basic strain obtained in Example 1. The PDH enzyme activity was measured under aerobic conditions and anaerobic conditions. 21% oxygen was included in the air under aerobic conditions, while 0% oxygen was included in the air under anaerobic conditions. The PDH activity was measured by the common PDH activity measurement method wherein the strain is cultured for two hours under the oxygen conditions described above and then variation of NADH concentration is measured using pyruvate and NAD+ as substrates.

To equalize the protein expression levels, the C. glutamicum cells cultured under aerobic conditions were divided into a cell quantity that is the same as that under anaerobic conditions. Then, the cells were further cultured at 30° C. for two hours with one of the flasks kept under oxygen-free conditions by injecting nitrogen.

The microorganisms cultured under each oxygen condition were immediately cooled with ice and obtained by centrifugation. The yielded microorganisms were pulverized by using 0.1 mm silica gel beads, and then the whole proteins were obtained by immediately centrifuging the pulverized cell suspension.

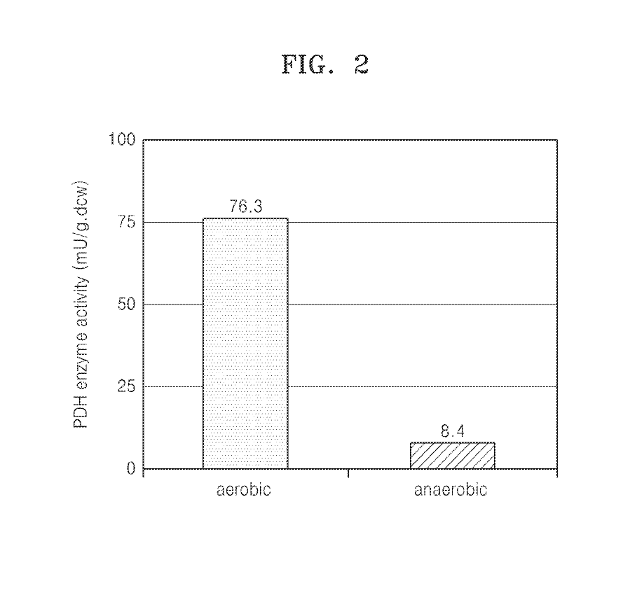

The protein activity was measured by measuring the absorbance at 25° C. at 340 nm wavelength using a thermo UV spectrometer. A reaction mixture was prepared by adding 2.5 mM NAD, 0.2 mM thiamin pyrophosphate, 0.1 mM coenzyme A, 0.3 mM dithiothreitol, 5 mM pyruvate, 1 mM magnesium chloride, and 1 mg/ml BSA (Bovine serum albumin) to 0.05 M potassium phosphate buffer (pH 7.8). The result shows that PDH enzyme activity under anaerobic conditions was about 11% of that under aerobic conditions (FIG. 2).

### Example 6

### Measurement of E. coli PDH Protein Activity Depending on Oxygen Conditions

PDH activity in C. glutamicum strains in which MD0375, MD0376, or MD0377 vector was introduced, as prepared in Example 4 was measured under anaerobic conditions. The PDH enzyme activity was measured under aerobic conditions and under anaerobic conditions. 21% oxygen was included in the air under aerobic conditions, while 0% oxygen was included in the air under anaerobic conditions. The PDH activity was measured after culturing the microorganisms under the respective oxygen conditions by measuring a decrease in pyruvate which was a substrate of the enzyme.

The protein activity was measured by measuring the absorbance at 25° C. at 340 nm wavelength using a kinetic spectrometer (Thermo Scientifics). A reaction mixture was prepared by adding 2.5 mM NAD, 0.2 mM thiamin pyrophosphate, 0.1 mM coenzyme A, 0.3 mM dithiothreitol, 5 mM pyruvate, 1 mM magnesium chloride, and 1 mg/ml BSA (Bovine serum albumin) to 0.05 M potassium phosphate buffer (pH 7.8).

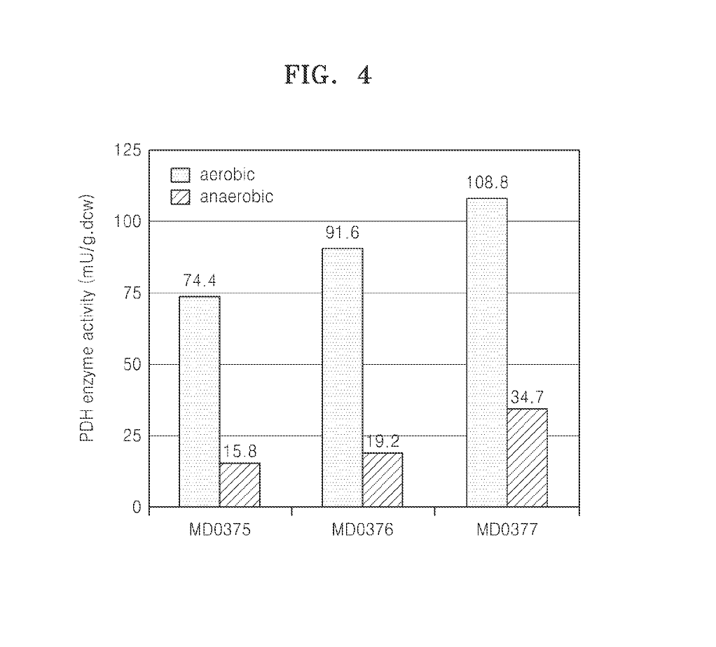

The result shows that the PDH activity was 15.8 mU/g and 19.2 mU/g in the strain in which the MD0375 vector was introduced and in the strain in which the MD0376 vector was introduced, respectively. On the contrary, the PDH activity was 34.7 mU/g in the strain in which the vector MD0377 (FIG. 4).

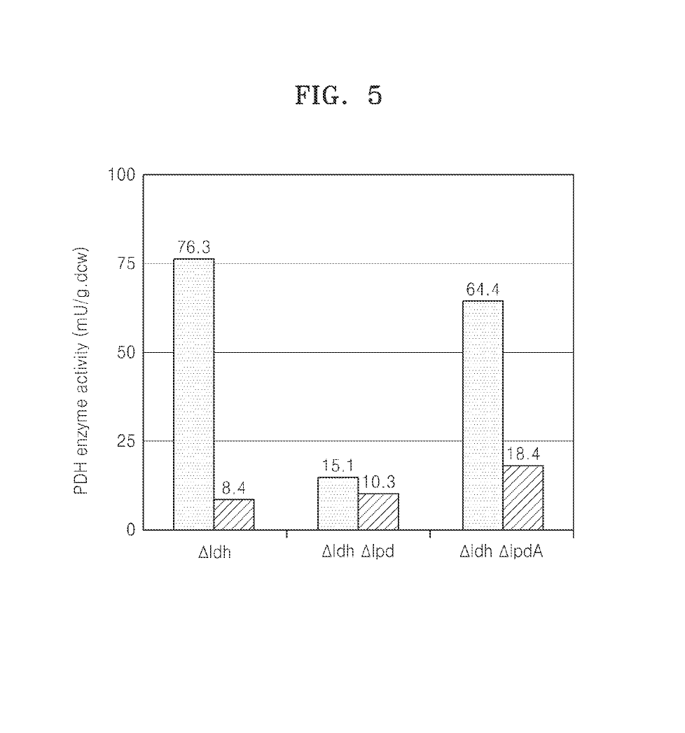

In addition, in order to verify lpd gene closely associated with PDH activity in a Corynebacterium, each of NCgI0355 (lpd) and NCgI0658 (lpdA) was deleted in genome of the basic strain of Example 1. The PDH activity of CGL (Δldh, Δlpd) was greatly decreased under anaerobic conditions and even under aerobic conditions compared to that of CGL (Δldh, ΔlpdA), which the PDH activity under aerobic conditions and under anaerobic conditions was not significantly different from that of the basic strain (FIG. 5). The result shows that the NCgI0355 gene (lpd) was directly associated with the PDH activity in a Corynebacterium.

### Example 7

### Measurement of 1,4-BDO Production of Microorganism Including PDH of which Activity is Maintained Under Anaerobic Conditions

The prepared microorganism including the PDH vector was cultured by a fermentation process. The 1,4-BDO production was measured every three hours while culturing the 1,4-BDO producing CGL strains including the PDH vector and the control group vector in a fermentation culture medium for amino acid production (glucose 40 g/L, corn steep liquor 10 g/L, ammonium sulfate 2 g/L, potassium phosphate 1 g/L, iron sulfate 10 mg/L, manganese sulfate 10 mg/L, zinc sulfate 0.1 mg/L, copper sulfate 0.1 mg/L, thiamine HCl 3 mg/L, biotin 0.3 mg/L, Ca pantothenate 1 mg/L, and nicotinamide 5 mg/L) at 30° C. and at pH 7.0 under an aeration condition of 40 vvm (volume/volume per minute).

The result shows that the Corynebacterium including the mutation lpd^{E354K} showed 1,4-BDO production (5.51 g/L) 177% higher than that of the control strain (1.98 g/L). In the case wherein the PDH vector was introduced to the C058 strain incapable of producing 1,4-BDO, 1,4-BDO production was 0.27 g/L with wild type PDH vector and 0.35 g/L with the PDH vector including the mutation lpd^{E354K}.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A genetically modified microorganism comprising a polynucleotide encoding a pyruvate dehydrogenase that remains active or has increased activity under anaerobic conditions compared to pyruvate dehydrogenase of an unmodified microorganism of the same type.

2. The genetically modified microorganism of claim 1, wherein the activity of the pyruvate dehydrogenase under anaerobic conditions is more than 30% of the activity of the pyruvate dehydrogense under aerobic condition.

3. The genetically modified microorganism of claim 2, wherein the microorganism comprises a polynucleotide encoding pyruvate dehydrogenase (E1) protein, a polynucleotide encoding dihydrolipoyl transacetylase (E2) protein, and a polynucleotide encoding a mutant of dihydrolipoyl dehydrogenase (E3) protein.

4. The genetically modified microorganism of claim 3, wherein the pyruvate dehydrogenase (E1) protein comprises the amino acid sequence of SEQ ID NO: 1.

5. The genetically modified microorganism of claim 3, wherein the dihydrolipoyl transacetylase (E2) protein comprises the amino acid sequence of SEQ ID NO: 3.

6. The genetically modified microorganism of claim 3, wherein the mutant of dihydrolipoyl dehydrogenase (E3) protein comprises the amino acid sequence of SEQ ID NO: 9.

7. The genetically modified microorganism of claim 3, wherein the polynucleotide encoding a mutant of dihydrolipoyl dehydrogenase (E3) protein comprises the nucleotide sequence of SEQ ID NO: 10.

8. The genetically modified microorganism of claim 1, wherein the microorganism produces 1,4-butanediol (BDO).

9. The genetically modified microorganism of claim 1, wherein the microorganism is a Corynebacterium genus.

10. The genetically modified microorganism of claim 1, wherein the microorganism has no lactate dehydrogenase activity, or has decreased lactate dehydrogenase activity compared to an unmodified microorganism of the same type.

11. The genetically modified microorganism of claim 8, wherein the microorganism comprises a polynucleotide encoding 4-hydroxybutyrate (4HB) dehydrogenase comprising the amino acid sequence of SEQ ID NO: 11, a polynucleotide encoding 4-hydroxybutyryl CoA transferase comprising the amino acid sequence of SEQ ID NO: 12, a polynucleotide encoding alcohol dehydrogenase comprising the amino acid sequence of SEQ ID NO: 13, and a polynucleotide encoding CoA-dependent succinate semialdehyde dehydrogenase comprising the amino acid sequence of SEQ ID NO: 14.

12. The genetically modified microorganism of claim 11, wherein the microorganism additionally comprises a polynucleotide encoding succinyl CoA:coenzyme A transferase comprising the amino acid sequence of SEQ ID NO: 20.

13. A method of preparing a genetically modified microorganism that produces 1,4-BDO under anaerobic conditions, the method comprising
introducing a polynucleotide encoding a pyruvate dehydrogenase that remains active or has increased activity under anaerobic conditions compared to an unmodified microorganism of the same type into a microorganism;
inactivating or decreasing lactate dehydrogenase activity;
and introducing a polynucleotide encoding CoA-dependent succinate semialdehyde dehydrogenase, a polynucleotide encoding 4HB dehydrogenase, a polynucleotide encoding 4-hydroxybutyryl CoA transferase, and a polynucleotide encoding alcohol dehydrogenase into the microorganism.

14. The method of claim 13, wherein the polynucleotide encoding the pyruvate dehydrogenase comprises a polynucleotide encoding pyruvate dehydrogenase (E1) protein, a polynucleotide encoding dihydrolipoyl transacetylase (E2) protein, and a polynucleotide encoding a mutant of dihydrolipoyl dehydrogenase (E3) protein.

15. The method of claim 14, wherein the mutant of dihydrolipoyl dehydrogenase (E3) protein comprises the amino acid sequence of SEQ ID NO: 9.

16. The method of claim 14, wherein the polynucleotide encoding a mutant of dihydrolipoyl dehydrogenase (E3) protein comprises the nucleotide sequence of SEQ ID NO: 10.

17. The method of claim 13, wherein the microorganism is a Corynebacterium genus.

18. A method of producing a C4-chemical comprising
culturing the genetically modified microorganism of claim 1 under anaerobic conditions in a cell culture medium, whereby the microorganism produces a C4-chemical; and
recovering the C4-chemical from a resulting culture solution.

19. The method of claim 18, wherein the C4-chemical is 1,4-BDO.
